SEQUENCE LISTING (37 sequences)

SEQ ID NO: 1 - DNA, 1001 bp, Homo sapiens
agtctaaggagttctccagctgagatattctgaaagttgatagagagttgatagaacaatcttttcataaggtataaattctatctatttttccctaaaaacaaacaaacagcaaccattcttgcttctaattgggcagtacaatctgataggttggctagagacttgcagtggggtgtccctggtacctattcaaagactgtagctttcttctatctcattctcattttctattctttgcattgtagagttttggagcaaagaaggtcatcaaacttatacagtgagcctaacagtttccttttaagatgaggaaactgagccccagccagccatgtgattcatcacagttccttggtggctgagttgggaggagaacacacatcttctcagctcctcccactgctctttccattaagacagacagcctctcattcaaagtaagagaatttccatcatatgagcaagggacaatgagagaactgcttctcagtactcccygcttcttcctcacctacttcctcttcactggatttgtcaactcacctgtctttacgcaatagttacaatgccagcatttctctacattacatacttcagcgattctcttactggctttgcaaagtcacccaaacacgaatggaaatagtaaggtagagtttcagccatgagttgagttcatagctttagcaactgttaaatagctaatgtctactttctggagaataaatgctttgcaagaccctcggcaatgaaaccaaagaaagaatttaaatagcctcaccgaataattagtcagcttttcgaagtcatctcgtttctttttgttgctattgaaaaacttgacattcatgtcttccttgatggtctccacactcttttggatgctctggtcatctttaaagtttttaaaaagtttgaagtaaaaggagacaatttggctctgcattatttttctgtcactctccttggaaggaaagagcacaaacagaggatgatgt

SEQ ID NO: 2 - DNA, 811 bp, Homo sapiens
ctgggttacttcccttttcaaatactcagtaacattcttgtttgcaataagacagaataagcaccacttattttgttccagatgatgtattgcactgtaagcaaaaatgcttctggtttttatttttaaagtcttccttatttttccccctacagtgctcattgagggcaggatttggcccccagctccttctggaacccaatttccttccatgttccctatcacaaccctacagacactggcctccttgctgttctcaaatactcttgacacatccttgctcagagactttgctgttcccacatttggaattctctgccctgagatgtttgaatgtctgccttcctgctcatgcagctgctgcccaaaggtcacctcttcagatagcctttgcctccaccttctctcccacttatctcaatcctgctcaattgttcttcccagctgttgtcactatttaccattacaccacacattaacacatgttgtttatcatctggmtcccttgccagaacacaggccctgtgaggatatgggaacttttctgtttgattcccaccatgtgcctggcatgaagtatttccttttaaatacttatcaaatgacggaatttcaactccaaggagaacctagaggcctagtgtagataaaaatgaacatagtagattttagctggtttaagaaactaatccaaccttcacttgaggatcgtaactcagagaaggaagacaggcattctttaatcgatagattgcttcagatgcaaagaggcaagtaaaggaaagaagttggttaaaaaataaaataaagt

SEQ ID NO: 3 - DNA, 2391 bp, Homo sapiens
atctacattgtgaaaagaccttctccacctttcctatttactttcactatgctcaaccacactggctgcatttcagatccccaaataagcctcctattctgcccaggaccaccccacagtctgctgcctctgttcagacagctctgcacacctcctgcctggccagctccctgtcatctggccacttcctatgtgtcaccttcccctataatatgctccttccccaggctaccttaggtgcctatcttagtctttccagcaccctatggtccaccatacttttagagcatttttcacagtttgtaattgtatatttagttgtataattctttgctttacctttcccctaaaggactgtaagttcatgggggcaggaatgggtctgttttgcccatcacatatctccaacacctagcaaaggcctggtacatattttgcactcaaaaaatgcaagctgagaaaatgagtgcatgagaacagctcaaggccatcctctcccaggaagccttcctcacttatgcccaggccactgggttacttcccttttcaaatactcagtaacattcttgtttgcaataagacagaataagcaccacttattttgttccagatgatgtattgcactgtaagcaaaaatgcttctggtttttatttttaaagtcttccttatttttccccctacagtgctcattgagggcaggatttggcccccagctccttctggaacccaatttccttccatgttccctatcacaaccctacagacactggcctccttgctgttctcaaatactcttgacacatccttgctcagagactttgctgttcccacatttggaattctctgccctgagatgtttgaatgtctgccttcctgctcatgcagctgctgcccaaaggtcacctcttcagatagcctttgcctccaccttctctcccacttatctcaatcctgctcaattgttcttcccagctgttgtcactatttaccattacaccacacattaacacatgttgtttatcatctggctcccttgccagaacacaggccctgtgaggatatgggaacttttctgtttgattcccaccatgtgcctggcatgaagtatttccttttaaatacttatcaaatgacggaatttcaactccaaggagaacctagaggcctagtgtagataaaaatgaacatagtagattttagctggtttaagaaactaatccaaccttcacttgaggatcgtaactcagagaaggaagacaggcattctttaatcgatagattgcttcagatgcaaagaggcaagtaaaggaaagaagttggttaaaaaataaaataaagtagagaatagtggagaagagaaactaaagtggatgaaagacaaaaatatgaaaattgcaaggatcagaaatcctttcagtagctatgtgaccaccagaaagtcaaagtcctgacttgctcctcctcctgacaaaggtggctctgaatgaagggcaacctctccatgccggcaaatccgctcaatgttcatgcattcgatgaagtatgccacacaagatagtaatcagaggaaactcccaaaaatcaaaataaaaagaattaaaacagtgaagaaagaattatggataataataaaaaagaaattagatgataatggggaaacatataacagtctcacaatgaaaagaatatttcctktattaaacataactggtattacatcattacatcaaacgggttgacctgttcagagttcccaaaaagaaaagggtataagatgttgtcacgttgaaaaccaccccaaatctataacttgagatttgcatatatatatgaaaacataaacaagttgggtgtgttaaggccttgttgtaacagagcagtaagaactgtaataaattggggtgggtgggaggtttgctgctgattgtctgactcagaaaacaagcccttaatcagttcttgaccagccctgtgttttgattgcccaacataagattagaattatgttggcagtttaatattcaattgttgaggagaaaatagctatgtctataatatcacatccggtctcagaatctgcaaccctctgaaacagtctggcttctaccattagccattactttgaccttagctgatcaaacttcttgtatctcaaaaattaacttctatcagaaatgagttcttaaaattcttattgacttccttgataaactaataattcagactacacgaatctgacaataaaattgagaaaagtccaggcacagtggctcatgcctataatgccagcactttggaaggcagaggaaggaggattgccggaggtcaggagtttgagaccagcctggtcaacacagcaagactctgtctctacaaaaaaaaaaaaaaaa

SEQ ID NO: 4 - DNA, 601 bp, Homo sapiens
gaagcaagaggttggagtgatttaaggaagtgggagatcaggaagtgttaagcacaaagttaacttatacctttaaaaaaaaaggaaaattaaagacaacaagcagatatatacattatataaataaacacaagaacttgttaaacttgtgaaacacaggactacccaagacaaatgtttccctttccatttctactctaggctatatgaggaatctatttttctctttttcttagaactcttactctgcaggatcaaaatgtcaagcagtgaccacataagtgggaagtagatcaagacrtgcttataattacattcttgtggttggtataaaattctgtaccacagccattccttagaagccagctcatgggatcaaacagacaagcaagctcacttctcaagcccacgatgttacgtattagtccatattggtgagttcctttgtgcccaaacttagctgccgcacactacagtgataggaaactgccaacagttttttgtcaaacccactacagtggatcccacaatgggctgagacctcgtctagagtcatcaggttttccaggatgtgcagatggcctggaaacttggccaagag

SEQ ID NO: 5 - DNA, 970 bp, Homo sapiens
tgtttcccatggtctacctgataaacatacttcaaagatatgtctgcccttgctcttggtctccactgtcaccactccagtctaagccagcatcatgctatcctgacctcctacagtgccctccaacctagtcctttgattcttcttttgacctcttccaatacctttccatacacagcttgaagcatgagcatcttaaatcatcaatctgatgatgtcactccccttcttaaaaccttttgttgtttcccagtattttaaggataaaatccaaaatccaaagtcccacatgttctggatgcatctacattgtgaaaagaccttctccacctttcctatttactttcactatgctcaaccacactggctgcatttcagatccccaaataagcctcctattctgcccaggaccaccccacagtctgctgcctctgttcagacagctctgcacacctcctgcctggccagctccctgtcatctggccacttcctatgtgtcaycttcccctataatatgctccttccccaggctaccttaggtgcctatcttagtctttccagcaccctatggtccaccatacttttagagcatttttcacagtttgtaattgtatatttagttgtataattctttgctttacctttcccctaaaggactgtaagttcatgggggcaggaatgggtctgttttgcccatcacatatctccaacacctagcaaaggcctggtacatattttgcactcaaaaaatgcaagctgagaaaatgagtgcatgagaacagctcaaggccatcctctcccaggaagccttcctcacttatgcccaggccactgggttacttcccttttcaaatactcagtaacattcttgtttgcaataagacagaataagcaccacttattttgttccagatgatgtattgcactgtaagcaaaaatgcttctggtttttatttttaaagtcttcctt

SEQ ID NO: 6 - DNA, 701 bp, Homo sapiens
ggagaaaatagctatgtctataatatcacatccggtctcagaatctgcaaccctctgaaacagtctggcttctaccattagccattactttgaccttagctgatcaaacttcttgtatctcaaaaattaacttctatcagaaatgagttcttaaaattcttattgacttccttgataaactaataattcagactacacgartctgacaataaaattgagaaaagtccaggcacagtggctcatgcctataatgccagcactttggaaggcagaggaaggaggattgccggaggtcaggagtttgagaccagcctggtcaacacagcaagactctgtctctacaaaaaaaaaaaaaaaaaaaatagaaataaggctgggcgcggtggctcatgcctgtaatcccagcactttgggaggccgaggcgggcggatcacaaggtcaggagatcgagaccatcctggctaccacggtgaaaccctgtctctactaaaaatacaaaaaattagccaggcgtggtagcgggtgcctgtagtcctagctacttgggaggctgaggcaggagaatggcatgaaccagggaggcagagcttgcagtgagccaagatcaggtcactgcactccagcctgggcgacagagcgagactctttctcaaaaataaaataaaataaaataaaataaaataaaataaaataaaataaa

SEQ ID NO: 7 - DNA, 601 bp, Homo sapiens
gaggatgaaatttgttactataaataaaatgcactgcctgacatatgcactacataagcattgcaattattataattgattccaaactcattgatgtctttatttcagtcttcctctccaccgagtgtgtgtgtgtgtgtgtgtgtgtgtgtgtgtgtgtgtgtgtgttgaaacttaacatctggaatggaggtggaaggcaagactgagagattggagatggcgtcagagagctcctagcaggtatgtcaaggctctgatccaatctctgggctagcagaagcctccagcagctctctgrggcaggagcataaggaaactctaagatctgtacttctgctttagcactaaaccagtacaatgtagtctcccaaccaggaaatggatggagactccaactcagatgctgtagatctttgctaaggccattcaatccattcacaatagagtcaaaatgagatctctggaagttctgaaaactcttattgtgattcaggtcacagaattaattttctgatcctataagctcaatatctatgtcctatcttgaaccctttatccacttttatgtacataaatctttgtctttatcagtgactat

SEQ ID NO: 8 - DNA, 881 bp, Homo sapiens
ctctttagtaagctgtatatccaccaaatgctgtgacacaaggaatgggcctcaaaatgtttcaggccagcacagctttgtctttgcaagtcatttttcagtggcagttaaatattgtggcaaagccaaggtacagactcacgtgcattaaaatatgcttatgagtaataataccataagtatttccatgtgcattaggtgtcaagaacttcacacacatcatttcattcccttatcatggcactcagtctggtgggtattactgtatctgtttcacaataggaaacccagtatctgagatgttcagtgcctttcccagttacccagttggtaagtgggaaagtcaggacatgaattttcagtgattttattaaaaaggcaggatgatacagtgatttaaaggcctaattctagacccaggttgcttagattcaagtcccagctctgccatactctgttagtataactttggcmaagtcactatgcctcagtttcctcatctgtaaaatggggatcataatagtatctaccttatggtactattattttatttattgttattgtgaggatgaaatttgttactataaataaaatgcactgcctgacatatgcactatataagcattgcaattattataattgattccaaactcattgatgtctttatttcagtcttcctctccaccgagtgtgtgtgtgtgtgtgtgtgtgtgtgtgtgtgtgtgtgtgtgttgaaacttaacatctggaatggaggtggaaggcaagactgagagattggagatggcgtcagagagctcctagcaggtatgtcaaggctctgatccaatctctgggctagcagaagcctccagcagctctctggggcaggagcataagga

SEQ ID NO: 9 - DNA, 701 bp, Homo sapiens
gccatacttcttaaatcaactctatatgtgatcctcagagtgatacttctaagacacaaatttgatcaaccatggtaggctaaataatggctcccccaaagatatttacatcctaccctgtgaatatgctaccttacatggaaaaaggggctttaaaaatatgattgagttgagaatcttgagatggaaagttatccagtattatctgagtgggcccaatgtcctcacaagggtccttataaagtgagaaataagaggatcaaagtaagtagtaggtgatgtgacagcagaagcaagaggttggagtgatttaaggaagtgggagatcaggaagtgttaagcacaaagttaacttatacctttaaaaaaaaaggaaaattaaagacaacaagcagatatatacattatataaataaacacaagaacttgttaaacttgtgaaacacaggactacccaagacaaatgtttccctttccatttctactctaggctatatgagraatctatttttctctttttcttagaactcttactctgcaggatcaaaatgtcaagcagtgaccacataagtgggaagtagatcaagacgtgcttataattacattcttgtggttggtataaaattctgtaccacagccattccttagaagccagctcatgggatcaaacagacaagcaagctcacttctcaagcccacga

SEQ ID NO: 10 - DNA, 601 bp, Homo sapiens
caaggtaattcaatctgacattatctgtccctgtactgcaaagatttccgtttttcaatcatgatacttagtggtgggtaggtgtggcagagccagtgcgaattgacacagcttttctggaaagctgtttgcagtatggatgatgagccttaaaatgttaaatccttgtacttaatgctatttttaggggtacactctaaaatagtcactgataaagacaaagatttatgtacataaaagtggataaagggttcaagataggacatagatattgagcttataggatcagaaaattaattcygtgacctgaatcacaataagagttttcagaacttccagagatctcattttgactctattgtgaatggattgaatggccttagcaaagatctacagcatctgagttggagtctccatccatttcctggttgggagactacattgtactggtttagtgctaaagcagaagtacagatcttagagtttccttatgctcctgccccagagagctgctggaggcttctgctagcccagagattggatcagagccttgacatacctgctaggagctctctgacgccatctccaatctctcagtctt

SEQ ID NO: 11 - DNA, 1000 bp, Homo sapiens
gtctcttcacttcaatgacatctctatttcctcccttctctgtcctgttacccagccattcttatcaaatgttggagtctccaagattctattcttgaacttcttttcctactcaatgtgttctccccagatgatctctttcttatggcagtttcaggcctgaatttccaatgacaattgttatcaacaacatgatattttatcagccactcaaactgtgtggctcaaactgaactaattgtcccctgaccacaaattctcatgcattccttgcaggttaattatatcacaacaaactaactgaaatcaaaaattgggagtcaacctcgattctttcctctcctttatttcctaccggtcgtgaaatctttgctaatttaaactcctggatatctttggaattcatatcctcctttccctcctcattgtcatatctttcttcaaagttgtgcctgtattattgcaacattttaaaatttattttcctgaacttctgccatrcttcttaaatcaactctatatgtgatcctcagagtgatacttctaagacacaaatttgatcaaccatggtaggctaaataatggctcccccaaagatatttacatcctaccctgtgaatatgctaccttacatggaaaaaggggctttaaaaatatgattgagttgagaatcttgagatggaaagttatccagtattatctgagtgggcccaatgtcctcacaagggtccttataaagtgagaaataagaggatcaaagtaagtagtaggtgatgtgacagcagaagcaagaggttggagtgatttaaggaagtgggagatcaggaagtgttaagcacaaagttaacttatacctttaaaaaaaaaggaaaattaaagacaacaagcagatatatacattatataaataaacacaagaacttgttaaacttgtgaaacacaggactacccaagacaaatgtttccctttccatttctactctaggctatatgaggaatc

SEQ ID NO: 12 - DNA, 601 bp, Homo sapiens
ttcccttgtcttaattgtctacctccccaatggctttcccttaaattataaaatgtgaaatccactgccatcactaggggaaacctgcttctctctgtattgactatccaattttttccctctgtttacaatcacatttcttgaataataacttcactctcaccctcaatacttccatcagctccaggcttcatgggctctctaaaattgttcttaataagatccaacattaccttctatctgttcgcttagtccaatagatcgtcaggcctgatcttacttggctccgtaagatatctgrcatcacagccaacaccttccttcttgaaagcctctcgtctcttcacttcaatgacatctctatttcctcccttctctgtcctgttacccagccattcttatcaaatgttggagtctccaagattctattcttgaacttcttttcctactcaatgtgttctccccagatgatctctttcttatggcagtttcaggcctgaatttccaatgacaattgttatcaacaacatgatattttatcagccactcaaactgtgtggctcaaactgaactaattgtcccctgaccacaaattctcatg

SEQ ID NO: 13 - DNA, 761 bp, Homo sapiens
gagcacagctaagctactacttgagcacagctactttctcttgtagctttatcccaaaccatggcttcagctgccactcatacccaaagggttcccagctttatattccagccttgatttccactgccaattagctagtactgggtatcccacaggtgattgtaattcatcctgttcagacctgaactcagcatgttttctttaaacttgcccatccttctaggtccttatcccaatgaatagaactatagtccacttacatgtccatgaacctggagttaccttagatagtccctttctctacccaacctccgtgtataatcaacctacaatcttttgtacctgttacatactcttttctctcaacctgtgctatcataggcttcacacaagctctcattacctctcacctgagataatatgatggagatctaatttgttgacctgcttctagtcactcctctcaccctagcctcaaccatcttccatttggctgccagwgagtttgctcactggtttgtttttgtttcacacctgctcagaatctttcagtggttctcaaacatctaaggagattcacaatggcctgccttctgcctagctctccaggctaatgtaccatgacacagcctgtgctctagttatcgagatctttttgccattcctggagcacatcaaattctcttacatctttattctttcacacaggctctttcaccttactacctacacacaccaaggcatgtacacaaaccacagag

SEQ ID NO: 14 - DNA, 1001 bp, Homo sapiens
gaccctcgtctcaaccaaggggatccaaagaaacctgaaaatctagtttggatcatgatgggatggtggagtggggcaggggatgttgaacatgactcattatactctcctctttttggaattcaggcacaaaattgaccagcattaacattataacagagatcctaagactgacagaacagactctttatagccataagataccaaattctaacctgtctctagtacagcattacatgacagcaaagtcctgaaagaaaaaacatattttgaaatagcactgcaaacctgtctcttgtggaggaagttttacactctatagagattctccttcctttactaggtctctccagagagcctgacacctttgataagagatgttcacatctattctctctgaagcctgctacccagaggcttcatctacataacaagaacctaggcttctctagccctcccaacttgccttaactcaggctagtttcaactcttcaggcacarcttcaccctttcaaccaattgctaatcagaaaatctttgaatccacctacgaccagtaagccctgccacttcaagttgtcctgcctttccttattaaaccaatgtatatttcatacatattgattgatgtcttatgttccctaaaacacataaaaccaagatgtaacccaaccaccttcggccaatgttcctaggacctcctaataggtcataatccttaaccttggcaaaataaacttcaaaattgattgagacctgtctcagttactttttgacttatgacatcatctttacttgattatatatgcaaagaccttatttccaaataaggtcatattcacaggtaccaagggattagaactgtaacatattttacaattcaatccacagcaggccacataggaaacctcagtttcaaaacataggaatagtatagatcatattctttgatcataatgcattaaaattagaaattaataacaaaaataggaaaataaatc

SEQ ID NO: 15 - DNA, 702 bp, Homo sapiens
aagagaacatttgcaggggaactgccctttatgaaaccatcagatctcttgagacttatttaccaacacgagaacagcatggaaaaaacctgcccccgtgattcagttacctcccactggctcctccccacaacatgtggggattgtgggagctacaattcaagatgagatttgggtgaggacagagccaaaccatgtcacgcagtaataatgaaaagaaggaaagcaagaattgcctagattgttggcttaagcaattaataaataatgatgactttgactgagctgggaatgaggaaggggtgtgcagaaggtgagggtggacagagaatccagaattctctttagtttgagatgctaaatgaagacatccaggttgaaaggtcaactaggcagttgaatatagagtccctaaaataggactcactggtccctacatggtatctgataccatggcattgcatgagatgacatctaagagttagatggataagctaagargggccctgcctgtttacattagttattagcagatgagaaggctaaacaaataagagagcagagcaatacattttcatggcatacatatcagaaagtagagaataagtatcagattcccttccgtgttttagcagaaaaggatactaattggcaagtgtcaatttgatattgtcaccaagtcattcaaaagaatagaaacct

SEQ ID NO: 16 - DNA, 601 bp, Homo sapiens
aggatctggatgaggaaatagagatattctaggtaggcttttcccccatgctggactggaggtgaagagaagaaactgagaagaggagggcctagggtgtacagactaagctagcttatgattgatccctgacatgtaagcagccagcagtccaaaatcataattacaaaggggcatgtatgacaaacacagatgaagtttaagctgaccttgacatacaagagaagccaagagcaatccatttgtcagaagagaagagggttcacctggtagtcatagtgctcagagccaggtgagtgtsaagaatgctgttccagggtctgcctggtttaaaagtactctgaccagtgttgaggggctgaggatggccagacaggttgtcaagcttcattctaagcatgagtgggttcagaatagagctcccaaataggaaactgaagtaccaatactattttaaaatgggaatagcaccacccttcagatctgacaaccccagcaggggtagataagagacggtagcttctgtctgtgccaaagctggtcattcacttgcttagcatttggcttataataaaaaggtcagtgaagtcctaacactgtt

SEQ ID NO: 17 - DNA, 1001 bp, Homo sapiens
tcccgggcacaagcaatcctctcacctcagtctccctagtagttgagcctacagatgcacatcaccatacctggctaatttttctaatttttgtagagatggggttttgccatgttgcccaggctggtcttgaactcctggagaggagttaagcaaccacctgaggttgcccagctaggaagtggtggagctgggatccaaatctgtgcacatataccccagactgagcttgtgagctcagattgcagctcctgagtgaagagatgcttttaccacctacatcatagaaaacatggatgctgtcaagctcaacttccttcatctttgccatatccccttttaatgtacctatctatattctcactcattacatatatgcctccttctccacttctgtctatagagctaacccttaaacgtgcgatcttcacttatctcctctaccacctgatactactcatctcctctctccatcaatctcttgttcccttgtcttaattktctacctccccaatggctttcccttaaattataaaatgtgaaatccactgccatcactaggggaaacctgcttctctctgtattgactatccaattttttccctctgtttacaatcacatttcttgaataataacttcactctcaccctcaatacttccatcagctccaggcttcatgggctctctaaaattgttcttaataagatccaacattaccttctatctgttcgcttagtccaatagatcgtcaggcctgatcttacttggctccgtaagatatctgacatcacagccaacaccttccttcttgaaagcctctcgtctcttcacttcaatgacatctctatttcctcccttctctgtcctgttacccagccattcttatcaaatgttggagtctccaagattctattcttgaacttcttttcctactcaatgtgttctccccagatgatctctttcttatggcagtttcaggcctgaatttccaatgacaattg

SEQ ID NO: 18 - DNA, 1001 bp, Homo sapiens
aagggagtgagagaaagagagagagagagagcaagctctctggtgtttcctcttttaaggtcactaatcccatcagaccaaggtccaactttcatgacctcatttaaccctaattaactccccaaagtttcatctccaaatactatcacattgggcattaaggcccatttgaatttcaagggacacaaatgttgagtccacaacatgcacgataaggcacatatgtcacttagtccttaagtcactaaataatatatattttaagaataaaaaatctaatgtcctaataattcaggtaatcaaaaatatgagtatattttatacatataaatgtttattttgtatgtaaataatatatataatacatatacatatatactatatatatgtatatgtatattttatatatgtaaaatattaagttcagttcctttatataaattaataagcatcaattaaatgatgagtcctttatattcatttttcagatgtcatacttgwgaaccacagaaatgaaatcttgctccagaacacacagctggttggtggcaaggccccgccagctgattaccagtccagtgccctttccagcaaaccacaccaaccttaactggtatgaatgcatctctacatatcagtcaataaggcatataataaccttgggtcatattagagtttgagggataagtcctaggttctacatcactgacctctaggagccaacacagatatcagagacctgagccctatctcaagacactacagacagtgatgtgttacactggcagacatccccaatgcaatggaagagggcttcaagcaggctcttcaaaaccttttccagactcatagatgctgctggaaggttcaggaactaattcactttgagcagaataggaaacatatagtgatccaagttcctaagaacccagtagccatttttaatggagacagacaccagagtttcaatgggggctaccaccaggaggagctcctgagag

SEQ ID NO: 19 - DNA, 2391 bp, Homo sapiens
atctacattgtgaaaagaccttctccacctttcctatttactttcactatgctcaaccacactggctgcatttcagatccccaaataagcctcctattctgcccaggaccaccccacagtctgctgcctctgttcagacagctctgcacacctcctgcctggccagctccctgtcatctggccacttcctatgtgtcaccttcccctataatatgctccttccccaggctaccttaggtgcctatcttagtctttccagcaccctatggtccaccatacttttagagcatttttcacagtttgtaattgtatatttagttgtataattctttgctttacctttcccctaaaggactgtaagttcatgggggcaggaatgggtctgttttgcccatcacatatctccaacacctagcaaaggcctggtacatattttgcactcaaaaaatgcaagctgagaaaatgagtgcatgagaacagctcaaggccatcctctcccaggaagccttcctcacttatgcccaggccactgggttacttcccttttcaaatactcagtaacattcttgtttgcaataagacagaataagcaccacttattttgttccagatgatgtattgcactgtaagcaaaaatgcttctggtttttatttttaaagtcttccttatttttccccctacagtgctcattgagggcaggatttggcccccagctccttctggaacccaatttccttccatgttccctatcacaaccctacagacactggcctccttgctgttctcaaatactcttgacacatccttgctcagagactttgctgttcccacatttggaattctctgccctgagatgtttgaatgtctgccttcctgctcatgcagctgctgcccaaaggtcacctcttcagatagcctttgcctccaccttctctcccacttatctcaatcctgctcaattgttcttcccagctgttgtcactatttaccattacaccacacattaacacatgttgtttatcatctggctcccttgccagaacacaggccctgtgaggatatgggaacttttctgtttgattcccaccatgtgcctggcatgaagtatttccttttaaatacttatcaaatgacggaatttcaactccaaggagaacctagaggcctagtgtagataaaaatgaacatagtagattttagctggtttaagaaactaatccaaccttcacttgaggatcgtaactcagagaaggaagacaggcattctttaatcgatagattgcttcagatgcaaagaggcaagtaaaggaaagaagttggttaaaaaataaaataaagtagagaatagtggagaagagaaactaaagtggatgaaagacaaaaatatgaaaattgcaaggatcagaaatcctttcagtagctatgtgaccaccagaaagtcaaagtcctgacttgctcctcctcctgacaaaggtggctctgaatgaagggcaacctctccatgccggcaaatccgctcaatgttcatgcattcgatgaagtatgccacacaagatagtaatcagaggaaactcccaaaaatcaaaataaaaagaattaaaacagtgaagaaagaattatggataataataaaaaagaaattagatgataatggggaaacatataacagtctcacaatgaaaagaatatttcctttattaaacataactggtattacatcattacatcaaacgggttgacctgttcagagttcccaaaaagaaaagggtataagatgttgtcacrttgaaaaccaccccaaatctataacttgagatttgcatatatatatgaaaacataaacaagttgggtgtgttaaggccttgttgtaacagagcagtaagaactgtaataaattggggtgggtgggaggtttgctgctgattgtctgactcagaaaacaagcccttaatcagttcttgaccagccctgtgttttgattgcccaacataagattagaattatgttggcagtttaatattcaattgttgaggagaaaatagctatgtctataatatcacatccggtctcagaatctgcaaccctctgaaacagtctggcttctaccattagccattactttgaccttagctgatcaaacttcttgtatctcaaaaattaacttctatcagaaatgagttcttaaaattcttattgacttccttgataaactaataattcagactacacgaatctgacaataaaattgagaaaagtccaggcacagtggctcatgcctataatgccagcactttggaaggcagaggaaggaggattgccggaggtcaggagtttgagaccagcctggtcaacacagcaagactctgtctctacaaaaaaaaaaaaaaaa

SEQ ID NO: 20 - DNA, 1001 bp, Homo sapiens
gtcttttcacaatgtagatgcatccagaacatgtgggactttggattttggattttatccttaaaatactgggaaacaacaaaaggttttaagaaggggagtgacatcatcagattgatgatttaagatgctcatgcttcaagctgtgtatggaaaggtattggaagaggtcaaaagaagaatcaaaggactaggttggagggcactgtaggaggtcaggatagcatgatgctggcttagactggagtggtgacagtggagaccaagagcaagggcagacatatctttgaagtatgtttatcaggtagaccatgggaaacacatggaattttgatttatctatgctgtaaaaaaattagaaatacaaagaaaacaagagcccccagcttacaagctcttaaattagaacaatgaaatataccttgaccaggaaaccaggcaatctcactggagagcttgcatgtcagcagtaattgtcttttattcctggaaagtatttgrgagtctgatttgctactttcatacaaattccaaaataatactgaacttattagtaaaatagtatacctgaactaaatagtatacctactgtgtgctaggaacagctgtaggtttggttttgggacacagtggggagtaaatagtcagacctccttcctcaaagagctggaattctgacaaaacttaaatttctgtgtaccttttaagaaatgtataattttagccaaaagataataaataagagtgatgtgttttttaaatacatcagtttgatataggaaagtataaaataatactttctgaatttttatgagacaacttcaaaagattctattagaaaatactttaaagtaaataaattaattggcaaacaacttactgggaaagcatttgccatcaaaataaccaaagaataattttattactataaaaagatcttgtagaaatcattaaataaagcaggatcccccaacagaaaaaatgtactgaaagtataaata

SEQ ID NO: 21 - DNA, 51 bp, Homo sapiens
  misc_feature (26)..(26): n is a, c, g, or t
ataattctttgctttacctttccccnaaaggactgtaagttcatgggggca

SEQ ID NO: 22 - DNA, 1001 bp, Homo sapiens
atgtattgcactgtaagcaaaaatgcttctggtttttatttttaaagtcttccttatttttccccctacagtgctcattgagggcaggatttggcccccagctccttctggaacccaatttccttccatgttccctatcacaaccctacagacactggcctccttgctgttctcaaatactcttgacacatccttgctcagagactttgctgttcccacatttggaattctctgccctgagatgtttgaatgtctgccttcctgctcatgcagctgctgcccaaaggtcacctcttcagatagcctttgcctccaccttctctcccacttatctcaatcctgctcaattgttcttcccagctgttgtcactatttaccattacaccacacattaacacatgttgtttatcatctggctcccttgccagaacacaggccctgtgaggatatgggaacttttctgtttgattcccaccatgtgcctggcatgaagtatttccytttaaatacttatcaaatgacggaatttcaactccaaggagaacctagaggcctagtgtagataaaaatgaacatagtagattttagctggtttaagaaactaatccaaccttcacttgaggatcgtaactcagagaaggaagacaggcattctttaatcgatagattgcttcagatgcaaagaggcaagtaaaggaaagaagttggttaaaaaataaaataaagtagagaatagtggagaagagaaactaaagtggatgaaagacaaaaatatgaaaattgcaaggatcagaaatcctttcagtagctatgtgaccaccagaaagtcaaagtcctgacttgctcctcctcctgacaaaggtggctctgaatgaagggcaacctctccatgccggcaaatccgctcaatgttcatgcattcgatgaagtatgccacacaagatagtaatcagaggaaactcccaaaaatcaaaataaaaagaattaaaacagtgaagaaa

SEQ ID NO: 23 - DNA, 1110 bp, Homo sapiens
tgcaagctctccagtgagattgcctggtttcctggtcaaggtatatttcattgttctaatttaagagcttgtaagctgggggctcttgttttctttgtatttctaatttttttacagcatagataaatcaaaattccatgtgtttcccatggtctacctgataaacatacttcaaagatatgtctgcccttgctcttggtctccactgtcaccactccagtctaagccagcatcatgctatcctgacctcctacagtgccctccaacctagtcctttgattcttcttttgacctcttccaatacctttccatacacagcttgaagcatgagcatcttaaatcatcaatctgatgatgtcactccccttcttaaaaccttttgttgtttcccagtattttaaggataaaatccaaaatccaaagtcccacatgttctggatgcatctacattgtgaaaagaccttctccacctttcctatttactttcactatgctcaaccacactggctgcatttcagatccccarataagcctcctattctgcccaggaccaccccacagtctgctgcctctgttcagacagctctgcacacctcctgcctggccagctccctgtcatctggccacttcctatgtgtcaccttcccctataatatgctccttccccaggctaccttaggtgcctatcttagtctttccagcaccctatggtccaccatacttttagagcatttttcacagtttgtaattgtatatttagttgtataattctttgctttacctttcccctaaaggactgtaagttcatgggggcaggaatgggtctgttttgcccatcacatatctccaacacctagcaaaggcctggtacatattttgcactcaaaaaatgcaagctgagaaaatgagtgcatgagaacagctcaaggccatcctctcccaggaagccttcctcacttatgcccaggccactgggttacttcccttttcaaatactcagtaacattcttgtttgcaataagacagaataagcaccacttattttgttccagatgatgtattgcactgtaagcaaaaatgcttctggtttttatttttaaagtcttcctt

SEQ ID NO: 24 - DNA, 1001 bp, Homo sapiens
ggaacccaatttccttccatgttccctatcacaaccctacagacactggcctccttgctgttctcaaatactcttgacacatccttgctcagagactttgctgttcccacatttggaattctctgccctgagatgtttgaatgtctgccttcctgctcatgcagctgctgcccaaaggtcacctcttcagatagcctttgcctccaccttctctcccacttatctcaatcctgctcaattgttcttcccagctgttgtcactatttaccattacaccacacattaacacatgttgtttatcatctggctcccttgccagaacacaggccctgtgaggatatgggaacttttctgtttgattcccaccatgtgcctggcatgaagtatttccttttaaatacttatcaaatgacggaatttcaactccaaggagaacctagaggcctagtgtagataaaaatgaacatagtagattttagctggtttaagaaactaatccarccttcacttgaggatcgtaactcagagaaggaagacaggcattctttaatcgatagattgcttcagatgcaaagaggcaagtaaaggaaagaagttggttaaaaaataaaataaagtagagaatagtggagaagagaaactaaagtggatgaaagacaaaaatatgaaaattgcaaggatcagaaatcctttcagtagctatgtgaccaccagaaagtcaaagtcctgacttgctcctcctcctgacaaaggtggctctgaatgaagggcaacctctccatgccggcaaatccgctcaatgttcatgcattcgatgaagtatgccacacaagatagtaatcagaggaaactcccaaaaatcaaaataaaaagaattaaaacagtgaagaaagaattatggataataataaaaaagaaattagatgataatggggaaacatataacagtctcacaatgaaaagaatatttcctttattaaacataactggtattacatcat

SEQ ID NO: 25 - DNA, 1001 bp, Homo sapiens
cagctcatgggatcaaacagacaagcaagctcacttctcaagcccacgatgttacgtattagtccatattggtgagttcctttgtgcccaaacttagctgccgcacactacagtgataggaaactgccaacagttttttgtcaaacccactacagtggatcccacaatgggctgagacctcgtctagagtcatcaggttttccaggatgtgcagatggcctggaaacttggccaagaggtagggatcagcactctttagtaagctgtatatccaccaaatgctgtgacacaaggaatgggcctcaaaatgtttcaggccagcacagctttgtctttgcaagtcatttttcagtggcagttaaatattgtggcaaagccaaggtacagactcacgtgcattaaaatatgcttatgagtaataataccataagtatttccatgtgcattaggtgtcaagaacttcacacacatcatttcattcccttatcatggcactcagtstggtgggtattactgtatctgtttcacaataggaaacccagtatctgagatgttcagtgcctttcccagttacccagttggtaagtgggaaagtcaggacatgaattttcagtgattttattaaaaaggcaggatgatacagtgatttaaaggcctaattctagacccaggttgcttagattcaagtcccagctctgccatactctgttagtataactttggccaagtcactatgcctcagtttcctcatctgtaaaatggggatcataatagtatctaccttatggtactattattttatttattgttattgtgaggatgaaatttgttactataaataaaatgcactgcctgacatatgcactatataagcattgcaattattataattgattccaaactcattgatgtctttatttcagtcttcctctccaccgagtgtgtgtgtgtgtgtgtgtgtgtgtgtgtgtgtgtgtgtgtgttgaaacttaacatctgga

SEQ ID NO: 26 - DNA, 1925 bp, Homo sapiens
actgtagtgggtttgacaaaaaactgttggcagtttcctatcactgtagtgtgcrgcagctaagtttgggcacaaaggaactcaccaatatggactaatacgtaacatcgtgggcttgagaagtgagcttgcttgtctgtttgatcccatgagctggcttctaaggaatggctgtggtacagaattttataccaaccacaagaatgtaattataagcacgtcttgatctacttcccacttatgtggtcactgcttgacattttgatcctgcagagtaagagttctaagaaaaagagaaaaatagattcctcatatagcctagagtagaaatggaaagggaaacatttgtcttgggtagtcctgtgtttcacaagtttaacaagttcttgtgtttatttatataatgtatatatctgcttgttgtctttaattttcctttttttttaaaggtataagttaactttgtgcttaacacttcctgatctcccacttccttaaatcactccaacctcttgcttctgctgtcacatcacctactacttactttgatcctcttatttctcactttataaggacccttgtgaggacattgggcccactcagataatactggataactttccatctcaagattctcaactcaatcatatttttaaagcccctttttccatgtaaggtagcatattcacagggtaggatgtaaatatctttgggggagccattatttagcctaccatggttgatcaaatttgtgtcttagaagtatcactctgaggatcacatatagagttgatttaagaagtatggcagaagttcaggaaaataaattttaaaatgttgcaataatacaggcacaactttgaagaaagatatgacaatgaggagggaaaggaggatatgaattccaaagatatccaggagtttaaattagcaaagatttcacgaccggtaggaaataaaggagaggaaagaatcgaggttgactcccaatttttgatttcagttagtttgttgtgatataattaacctgcaaggaatgcatgagaatttgtggtcaggggacaattagttcagtttgagccacacagtttgagtggctgataaaatatcatgttgttgataacaattgtcattggaaattcaggcctgaaactgccataagaaagagatcatctggggagaacacattgagtaggaaaagaagttcaagaatagaatcttggagactccaacatttgataagaatggctgggtaacaggacagagaagggaggaaatagagatgtcattgaagtgaagagacgagaggctttcaagaaggaaggtgttggctgtgatgtcagatatcttacggagccaagtaagatcaggcctgacgatctattggactaagcgaacagatagaaggtaatgttggatcttattaagaacaattttagagagcccatgaagcctggagctgatggaagtattgagggtgagagtgaagttattattcaagaaatgtgattgtaaacagagggaaaaaattggatagtcaatacagagagaagcaggtttcccctagtgatggcagtggatttcacattttataatttaagggaaagccattggggaggtagaaaattaagacaagggaacaagagattgatggagagaggagatgagtagtatcaggtggtagaggagataagtgaagatcgcacgtttaagggttagctctatagacagaagtggagaaggaggcatatatgtaatgagtgagaatatagataggtacattaaaaggggatatggcaaagatgaaggaagttgagcttgacagcatccatgttttctatgatgtaggtggtaaaagcatctcttcactcaggagctgcaatctgagctcacaagctcagtctggggtatatgtgcacagattt

SEQ ID NO: 27 - DNA, 1001 bp, Homo sapiens
caggatcaaaatgtcaagcagtgaccacataagtgggaagtagatcaagacgtgcttataattacattcttgtggttggtataaaattctgtaccacagccattccttagaagccagctcatgggatcaaacagacaagcaagctcacttctcaagcccacgatgttacgtattagtccatattggtgagttcctttgtgcccaaacttagctgccgcacactacagtgataggaaactgccaacagttttttgtcaaacccactacagtggatcccacaatgggctgagacctcgtctagagtcatcaggttttccaggatgtgcagatggcctggaaacttggccaagaggtagggatcagcactctttagtaagctgtatatccaccaaatgctgtgacacaaggaatgggcctcaaaatgtttcaggccagcacagctttgtctttgcaagtcatttttcagtggcagttaaatattgtggcaaagccaaggtacaractcacgtgcattaaaatatgcttatgagtaataataccataagtatttccatgtgcattaggtgtcaagaacttcacacacatcatttcattcccttatcatggcactcagtctggtgggtattactgtatctgtttcacaataggaaacccagtatctgagatgttcagtgcctttcccagttacccagttggtaagtgggaaagtcaggacatgaattttcagtgattttattaaaaaggcaggatgatacagtgatttaaaggcctaattctagacccaggttgcttagattcaagtcccagctctgccatactctgttagtataactttggccaagtcactatgcctcagtttcctcatctgtaaaatggggatcataatagtatctaccttatggtactattattttatttattgttattgtgaggatgaaatttgttactataaataaaatgcactgcctgacatatgcactatataagcattgcaattatta

SEQ ID NO: 28 - DNA, 1187 bp, Homo sapiens
aatgtgaaatccactgccatcactaggggaaacctgcttctctctgtattgactatccaattttttccctctgtttacaatcacatttcttgaataataacttcactctcaccctcaatacttccatcagctccaggcttcatgggctctctaaaattgttcttaataagatccaacattaccttctatctgttcgcttagtccaatagatcgtcaggcctgatcttacttggctccgtaagatatctgacatcacagccaacaccttccttcttgaaagcctctcgtctcttcacttcaatgacatctctatttcctcccttctctgtcctgttacccagccattcttatcaaatgttggagtctccaagattctattcttgaacttcttttcctactcaatgtgttctccccagatgatctctttcttatggcagtttcaggcctgaatttccaatgacaattgttatcaacaacatgatattttatcagccactcaaactgtgtggctcaaactgaactaattgtccccygaccacaaattctcatgcattccttgcaggttaattatatcacaacaaactaactgaaatcaaaaattgggagtcaacctcgattctttcctctcctttatttcctaccggtcgtgaaatctttgctaatttaaactcctggatatctttggaattcatatcctcctttccctcctcattgtcatatctttcttcaaagttgtgcctgtattattgcaacattttaaaatttattttcctgaacttctgccatacttcttaaatcaactctatatgtgatcctcagagtgatacttctaagacacaaatttgatcaaccatggtaggctaaataatggctcccccaaagatatttacatcctaccctgtgaatatgctaccttacatggaaaaaggggctttaaaaatatgattgagttgagaatcttgagatggaaagttatccagtattatctgagtgggcccaatgtcctcacaagggtccttataaagtgagaaataagaggatcaaagtaagtagtaggtgatgtgacagcagaagcaagaggttggagtgatttaaggaagtgggagatcaggaagtgttaagcacaaagttaacttatacctttaaaaaaaaaggaaaattaaagacaacaagcagatatatacatt

SEQ ID NO: 29 - DNA, 1001 bp, Homo sapiens
agtgatttaaaggcctaattctagacccaggttgcttagattcaagtcccagctctgccatactctgttagtataactttggccaagtcactatgcctcagtttcctcatctgtaaaatggggatcataatagtatctaccttatggtactattattttatttattgttattgtgaggatgaaatttgttactataaataaaatgcactgcctgacatatgcactatataagcattgcaattattataattgattccaaactcattgatgtctttatttcagtcttcctctccaccgagtgtgtgtgtgtgtgtgtgtgtgtgtgtgtgtgtgtgtgtgtgttgaaacttaacatctggaatggaggtggaaggcaagactgagagattggagatggcgtcagagagctcctagcaggtatgtcaaggctctgatccaatctctgggctagcagaagcctccagcagctctctggggcaggagcataaggaaactctaagmtctgtacttctgctttagcactaaaccagtacaatgtagtctcccaaccaggaaatggatggagactccaactcagatgctgtagatctttgctaaggccattcaatccattcacaatagagtcaaaatgagatctctggaagttctgaaaactcttattgtgattcaggtcacagaattaattttctgatcctataagctcaatatctatgtcctatcttgaaccctttatccacttttatgtacataaatctttgtctttatcagtgactattttagagtgtacccctaaaaatagcattaagtacaaggatttaacattttaaggctcatcatccatactgcaaacagctttccagaaaagctgtgtcaattcgcactggctctgccacacctacccaccactaagtatcatgattgaaaaacggaaatctttgcagtacagggacagataatgtcagattgaattaccttgtgtttcttttattactacagaggtc

SEQ ID NO: 30 - DNA, 1001 bp, Homo sapiens
aatgtttattttgtatgtaaataatatatataatacatatacatatatactatatatatgtatatgtatattttatatatgtaaaatattaagttcagttcctttatataaattaataagcatcaattaaatgatgagtcctttatattcatttttcagatgtcatacttgagaaccacagaaatgaaatcttgctccagaacacacagctggttggtggcaaggccccgccagctgattaccagtccagtgccctttccagcaaaccacaccaaccttaactggtatgaatgcatctctacatatcagtcaataaggcatataataaccttgggtcatattagagtttgagggataagtcctaggttctacatcactgacctctaggagccaacacagatatcagagacctgagccctatctcaagacactacagacagtgatgtgttacactggcagacatccccaatgcaatggaagagggcttcaagcaggctcttyaaaaccttttccagactcatagatgctgctggaaggttcaggaactaattcactttgagcagaataggaaacatatagtgatccaagttcctaagaacccagtagccatttttaatggagacagacaccagagtttcaatgggggctaccaccaggaggagctcctgagagcctctgccacacaagctgatggtaggagcccaggtgacaaaagagactgagggcacctcccaaggactactgcagagggtactgtcagaccccatgctgagggagaggggcagtggctgtgcctggccaggctagtgaccagagattatgtgtgttggttcttttctttcagagcatttatcgcaattgcaatcagttaattacattattatatggttaaagtctgtcttctgagctggatcataacccccacagtccacaacggtttctattatctttacatttcccccccaacaccttgaatagtatccagcccacagtaactgctc

SEQ ID NO: 31 - DNA, 1001 bp, Homo sapiens
tcaggaggctgaggcaggagaatctcttgaaccttggaggcggaggttgcagtgagccaagatcatacaactgcactccaacccaagcaacagagtgagactccatctcaaaaaaaaaaaaaaaaatctagagataaattaagccatatttaatgtctggacaccagaggatctagactggtgaaaactcacattgcatctgaggatagctagcctgctgcagtgattgatagagatgttttagttaagaccctgaactccttaccatgagatcacagatagccctcaggcttgaggatgcccaaatttagaaatcacaggatgaagatctcatacaatgaaatttataaaacatcctattagacctgcaggtataaatatgatatcaaactttctcccagatgtgaatttaaaacaagacctattatccgtggcatcaagctaaaataatatgtatggactacattctttaaataaattatgctatagttgaagatcagrtatatccaaaaggcacactgatacatattaaaactttaaacaactttttattggaagaagtcttaaaaagctaaagaaatgagtctaataagcactttgatttttgtttttttgagacaggatctcactctgtggcccaggctggagagcagtggtttagtcacagctcactgcagccttaacttcccgggcacaagcaatcctctcacctcagtctccctagtagttgagcctacagatgcacatcaccatacctggctaatttttctaatttttgtagagatggggttttgccatgttgcccaggctggtcttgaactcctggagaggagttaagcaaccacctgaggttgcccagctaggaagtggtggagctgggatccaaatctgtgcacatataccccagactgagcttgtgagctcagattgcagctcctgagtgaagagatgcttttaccacctacatcatagaaaacatggatgctgtcaagctcaacttc

SEQ ID NO: 32 - DNA, 1001 bp, Homo sapiens
ttgagctcaggagttcaagaccagcttggccaacatggtgaaaccccgtctctacaaaaaatacaaaaattggccaggcatggcggtgtatgcctgtagtcccagttactcaggaggctgaggcaggagaatctcttgaaccttggaggcggaggttgcagtgagccaagatcatacaactgcactccaacccaagcaacagagtgagactccatctcaaaaaaaaaaaaaaaaatctagagataaattaagccatatttaatgtctggacaccagaggatctagactggtgaaaactcacattgcatctgaggatagctagcctgctgcagtgattgatagagatgttttagttaagaccctgaactccttaccatgagatcacagatagccctcaggcttgaggatgcccaaatttagaaatcacaggatgaagatctcatacaatgaaatttataaaacatcctattagacctgcaggtataaatatgatatcaaacyttctcccagatgtgaatttaaaacaagacctattatccgtggcatcaagctaaaataatatgtatggactacattctttaaataaattatgctatagttgaagatcagatatatccaaaaggcacactgatacatattaaaactttaaacaactttttattggaagaagtcttaaaaagctaaagaaatgagtctaataagcactttgatttttgtttttttgagacaggatctcactctgtggcccaggctggagagcagtggtttagtcacagctcactgcagccttaacttcccgggcacaagcaatcctctcacctcagtctccctagtagttgagcctacagatgcacatcaccatacctggctaatttttctaatttttgtagagatggggttttgccatgttgcccaggctggtcttgaactcctggagaggagttaagcaaccacctgaggttgcccagctaggaagtggtggagctgggatccaaatctgt

SEQ ID NO: 33 - DNA, 1001 bp, Homo sapiens
gttgggctattgattgaaatgtcattgattctatagactaattagggaaggaatgacaactttaaaacactgagtctttttgtttataagcatttaagaacactgtatatttctccatttatttggcttcttttaacgtctttcaatatgtggctttatgattctctgtgtctttttgtaacagactttcagatggtctccatcctctagatttcgtgtgcttgtctttcctactcatgaatatccagtcaatttttctaaagtgaattttaccacattaactccaggttcaaaaatctgtggtaacttcctgttgcctatgggatcaaatacgagcttcgcaggctgggatttaaatggttctgtttcaccagccaacttcatgtcttgctagggctctactccatttgtttatttagggcagtattacagcatagaggtaaagagctcatttaggtaacagaatctaaataaaattagagtcaagccctgactccatcrcttactggcttatggcttagactgagggacttagtctaccggctcttccactataaaacaaagaaaacatgcatactcacagcataaggctcttgtaaagaataaatgagacagtgcatgtaagtcatgtagcacttgtgatttgctagcatgaattgctattgttgtgttagtattattattaacatattaaaaacataatagaatgtcttctctgagtgaataaccatcatcatacctatgtttaactcttttttgcctattaaaatgccctcctaaaagtggatttttgaatgctaagcaggatctggatgaggaaatagagatattctaggtaggcttttcccccatgctggactggaggtgaagagaagaaactgagaagaggagggcctagggtgtacagactaagctagcttatgattgatccctgacatgtaagcagccagccgtccaaaatcataattacaaaggggcatgtatgacaaacacagatgaag

SEQ ID NO: 34 - DNA, 1001 bp, Homo sapiens
gccaaagctggtcattcacttgcttagcatttggcttataataaaaaggtcagtgaagtcctaacactgttttatttttagtttcaccttaggaacatagtatttactctttatcttgatgatacaatgcaggtgacacatcacatggaatgacactatgtatgatgtgtaagagttggtcagacaatggtctgcccgggaaatctgaccatacataacaagaattcccaccagaggtaaacctgaatccataactagggaaggagggctcacaagaactaatgatggccctagagttctggtctttttagagatttatggttggtggactgccagtcatagcaaagggccctgagccaccagacagggagacaagcagaccaggatcgaggaagaagcttaagtctgcattctggacccacttaagagcaagctaggtaaacccctgactcaaagactgacagcatctatggctcacatcagacacagtgaatattgggwctggttagtgaagtagaaacattttcactccttagataatgatgtcactgggcttgacatttaggatgctatcaattaaccaaatatttatagctataaaggaaagaagataaaaattttaagtaagtggtgctgagtggagaaccatttatttgtttgctttttctctgtttgtgttaagataaaggagcctggagcatgtgtggagggtcaggagaatgatccatggaggatcctgaggaagccagaaacggggctgagcacagataggatgataagccctcaacagaagggaagaaacctcagtgccacaagggtgaatttagcatcacttcataaatcagaatggtcactaggactagggatatacagcagctgagggagaattgcagccagcgttctgttaatacaatggtggtgttctatcctgagcctggacaatgacaatttctcctatttgtctgggaatcatgatactaaaactcacctgacatggaatt

SEQ ID NO: 35 - DNA, 668 bp, Homo sapiens
gagattatgtgtgttggttcttttctttcagagcatttatcgcaattgcaatcagttaattacattattatatggttaaagtctgtcttctgagctggatcataacccccacagtccacaacggtttctattatctttacatttcccccccaacaccttgaatagtatccagcccacagtaactgctcaataagaagctgtttagttgagaaaaggaatgtcaactaccttttctaacaccttcaggatgaagaatgcacaaggaatgaaggttagaaagaggaaatagagattgctagtcttccatttcattcttccttgagcaatgtgtctcaaaatacratgaagttgaaagctctaaaatgggcggaaatttaagtaataacaacaacaacaataatacaaagctctaatttgcatctttaatgcattttattcttcccaagtaataacatatactcattttcatttggttttcatgatgtaacagttcctgtttatgataaggaaactgtcagagagattataactcctccagtatcaacaagctatgaaaatatgaaaccaggattcgaattcaggtctcacagttccacacccactattttttcacttcaactacattaaaacaataaaaaatattagtaaatgcaaatgtctgaaataaa

SEQ ID NO: 36 - DNA, 601 bp, Homo sapiens
agctgtttagttgagaaaaggaatgtcaactaccttttctaacacctttgggatgaagaatgcacaaggaatgaaggttagaaagaggaaatagagattgctagtcttccatttcattcttccttgagcaatgtgtctcaaaatacgatgaagttgaaagctctaaaatgggcggaaatttaagtaataacaacaacaacaataatacaaagctctaatttgcatctttaatgcattttattcttcccaagtaataacatatactcattttcatttggttttcatgatgtaacagttcctrtttatgataaggaaactgtaagagagattataactcctccagtatcaacaagctatgaaaatatgaaaccaggattcgaattcaggtctcacagttccacacccactattttttcacttcaactacattaaaacaataaaaaatattagtaaatgcaaatgtctgaaataaatgaggaaaatcaccattttaaaattagcaagctgcttctgtcttctagactagagatttggatttatctttgtggcagacagtcccgttgttctgggaaaaggaaaaagaaaacatatgcttgagagg

SEQ ID NO: 37 - DNA, 1001 bp, Homo sapiens
tgtgacaaagggacactgtgcttttattgcccctgagaattcctaaacactactgtaaccagaggcagtttagatacctccagcctccggcatcaaatcaaagagcaaagtactatagctcttttagactcttgggacctattagagcaggacatatgactgagggaagcttttcaagccttcataagcctttttaattacctcacattatttttcttagcctttgacttttccctattgttattagaataatctttaaaatgagagattttcattggagcaactctgtcctgctttaccagcatggtgggaaggagccaaggggcaggtaaagaggctctgtagactcagtcagtgatgtctctaccgaggatgaaaaattcgatgtgacctttggggttgtggcaaagataatgacactatccacctcataggaaggaggcaatagctaaattagttcatttatgtgaacaccacttaggacagagcttggcacataaytggggctctgaaaagttagttgcagttacttaggagtaatcatgatgctttgagagcagaaatatcttcttgattttcccccataaggtaaccgaaattaatttatatcccttttcagctgcctcccttaccatttcttacatatggtaactttcacttcttgttctccaaggtctccagtgcctctctctcaatgttttatctagatattacatatctgagggacacctagttagaaaatccttaagagacaatttaatatagattcacacttccttttctattaatgaagcattttcttctataaaattatttgctgcaattctcgaaagagtcaagtaatagcaaatgttgcaaaactttccagagtgaaatgaaggctagcttgaaaacttaacaaccattttgtattttcttggtaaaatgtatcttgttacaagtgataaaaatgaaatgagggagtttatgagaaatttctccctgacactgctacagtagtt

(19) United States
(12) Patent Application Publication
(10) Pub. No.: US 2018/0051078 A1
(43) Pub. Date: Feb. 22, 2018

(54) METHODS OF TREATING INFLAMMATORY BOWEL DISEASE WITH IFN-GAMMA THERAPY

(71) Applicant: Cedars-Sinai Medical Center, Los Angeles, CA (US)

(72) Inventors: Stephan R. TARGAN, Santa Monica, CA (US); Janine BILSBOROUGH, Simi Valley, CA (US); Dermot P. MCGOVERN, Los Angeles, CA (US)

(73) Assignee: Cedars-Sinai Medical Center, Los Angeles, CA (US)

(21) Appl. No.: 15/557,213

(22) PCT Filed: Mar. 15, 2016
(86) PCT No.: PCT/US16/22494

§ 371 Date: Sep. 11, 2017

Related U.S. Application Data
(60) Provisional application No. 62/133,841, filed on Mar. 16, 2015.

(51) Int. Cl. C07K 16/24; A61K 39/395

(52) U.S. Cl. CPC C07K 16/249; A61K 2039/505; A61K 39/395

(57) ABSTRACT

The present invention describes methods for treating inflammatory disorders of the gastrointestinal tract, including but not limited to Inflammatory Bowel Disease (IBD), Crohn's Disease (CD), Ulcerative Colitis (UC) and/or medically refractory ulcerative colitis (MR-UC) using anti-IFNG therapy. The present invention also describes a process for patient risk stratification.

### FIELD OF INVENTION

This invention relates to methods of treating inflammatory disorders of the gastrointestinal tract, including inflammatory bowel disease (IBD), Crohn's disease (CD) and ulcerative colitis (UC) and/or medically refractory ulcerative colitis (MR-UC), using anti-Interferon-Gamma (IFNG) therapy.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Inflammatory bowel disease (IBD) consists of Crohn's disease (CD) and ulcerative colitis (UC), the two common forms of IBD, which are chronic, relapsing inflammatory disorders of the gastrointestinal tract. CD and UC are thought to be related disorders that share some genetic susceptibility loci but differ at others.

Patients with UC demonstrate varying responses to medical therapies and need for surgery. Medically refractory ulcerative colitis (MR-UC) is a severe form of UC, which requires colectomy and remains a significant challenge in the management of IBD. Thus, there is a need in the art for the development of treatments for IBD, CD and UC, in particular, MR-UC.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of treating, preventing, reducing the severity of, reducing the likelihood of developing and/or reducing the likelihood of recurrence of inflammatory bowel disease in a subject, comprising: administering a therapeutically effective amount of said anti-IFNG therapy to the subject. In some embodiments, the anti-IFNG therapy can comprise fontolizumab. In other embodiments, the inflammatory bowel disease can be ulcerative colitis. In yet other embodiments, the inflammatory bowel disease can be medically refractory ulcerative colitis. In certain embodiments, the subject has been diagnosed with MR-UC. In certain other embodiments, the subject has been diagnosed with one or more risk variants, where the one or more risk variants can be rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 or a combination thereof. In various embodiments, the subject has an increase in IFNG secretion. In various other embodiments, the subject can have a decrease in IFNG methylation. In yet other embodiments, the subject can have an increased level of a serological factor. In some embodiments, the serological factor can be ANCA, ASCA, OmpC, I2, CBir or a combination thereof.

Various embodiments of the present invention provide for a process of identifying a subject in need of IFNG therapy, and optionally treating the subject, comprising: obtaining a biological sample from a subject; analyzing the sample for the presence or absence of one or more genetic risk variants; and identifying the subject in need of IFNG therapy if one or more genetic risk variants are present. In various embodiments, the subject has been diagnosed with IBD, CD and/or UC. In various other embodiments, the subject has been diagnosed with MR-UC. In various embodiments, the one or more genetic risk variants can be rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 and/or a combination thereof.

In various embodiments, the process can further comprise treating the subject identified with IFNG therapy. In some embodiments, the IFNG therapy can comprise fontolizumab. In various embodiments, the subject can have an increase in IFNG secretion. In various other embodiments, the subject can have a decrease in IFNG methylation. In yet other embodiments, the subject can have an increase in antibody reactivity to a serological factor. In certain embodiments, the serological factor can be ANCA, ASCA, OmpC, I2, CBir or a combination thereof.

Various embodiments of the present invention provide for a pharmaceutical composition for treating, preventing, reducing the severity of, reducing the likelihood of developing and/or reducing the likelihood of recurrence of inflammatory bowel disease in a subject, comprising: an anti-IFNG therapy; and a pharmaceutically acceptable carrier.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts an association of MR-UC with anti-microbial antibody levels in MR-UC versus non-MR-UC, in accordance with an embodiment of the invention (Prior Art).

FIG. 2 depicts the inverse correlation of IFNG DNA methylation with IFNG secretion in UC, in accordance with an embodiment of the invention (Prior Art).

FIG. 3 depicts decreased IFNG methylation is associated with increased antibody reactivity to microbial antigens in UC, in accordance with an embodiment of the invention (Prior Art). A) Quartile; B) Continuous regression analysis of UC or CD cohorts for four serological markers (ASCA, OMpC, I2 and CBir) was correlated with IFNG DNA methylation index.

FIG. 4 depicts IFNG secretion is decreased in individuals carrying the C allele at the methylation site rs1861794, in accordance with an embodiment of the invention (Prior Art).

FIG. 5A-5C depicts allele specific methylation in rs1861494 heterozygous IBD patients, in accordance with an embodiment of the invention (Prior Art). A) rs1861494; B) methylation percentage at +2052 and +2007; C) Correlation of methylation of rs1861494 with methylation index of IFNG promoter.

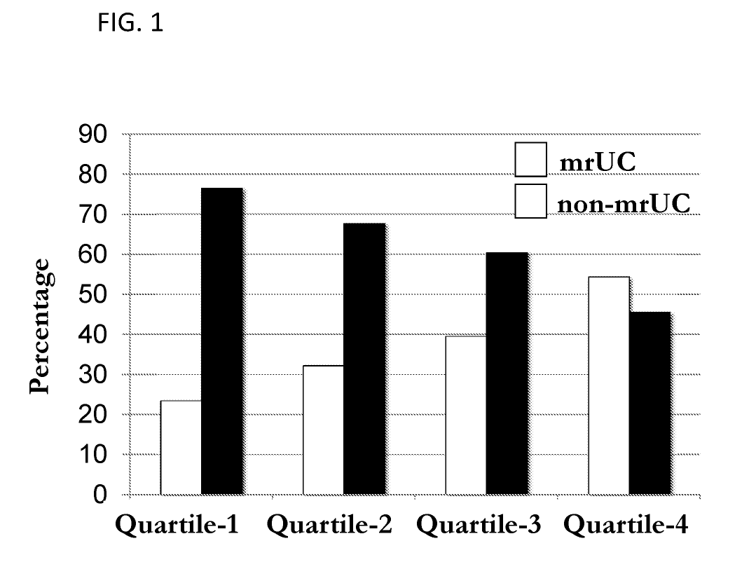

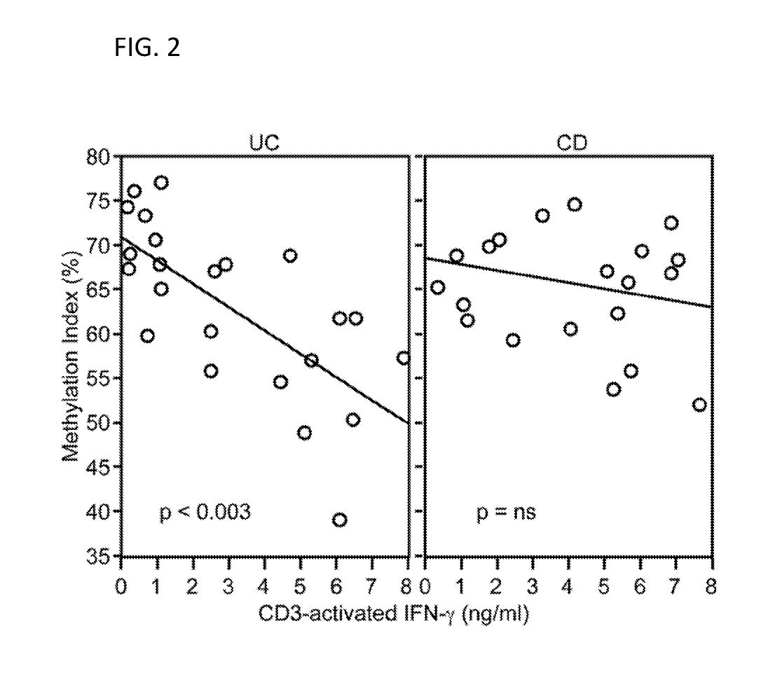

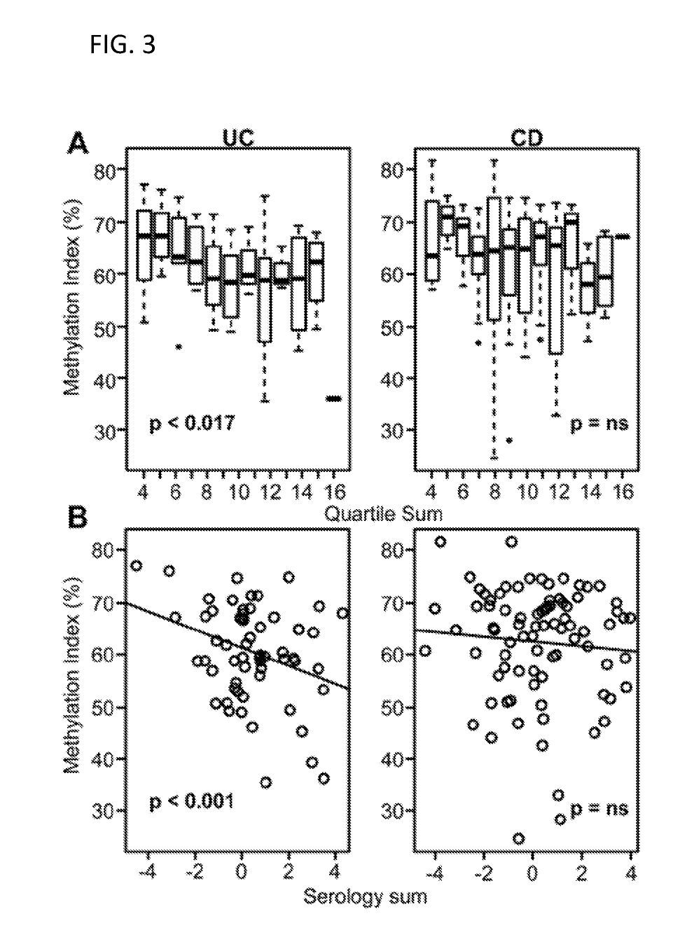

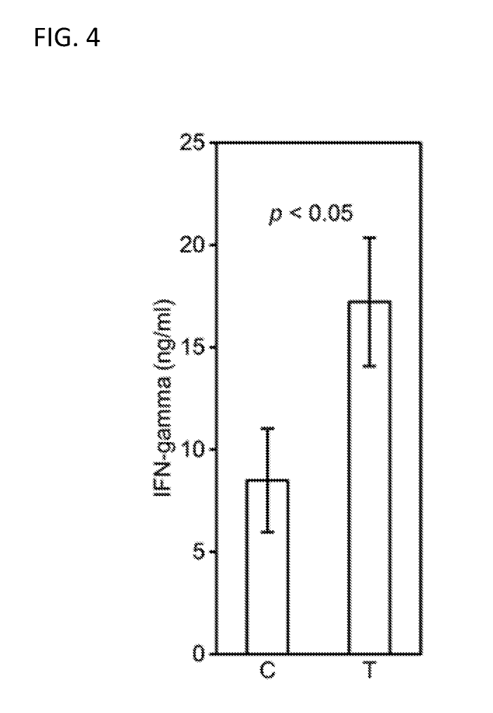

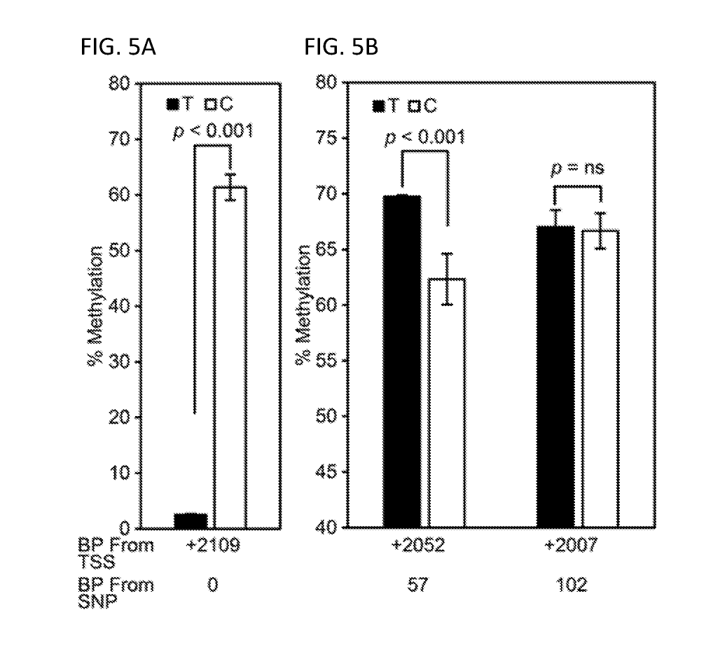

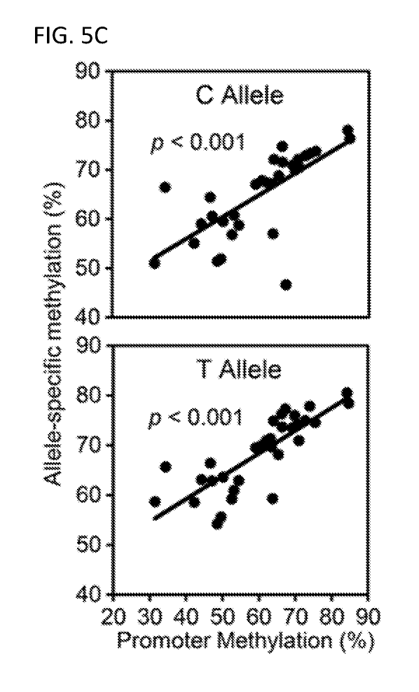

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“IBD”, “CD”, “UC” and “MR-UC” as used herein refer to Inflammatory Bowel Disease, Crohn's Disease, Ulcerative Colitis and Medically Refractive Ulcerative Colitis, respectively.

As used herein, “IBD” include “CD”, “UC” and/or “MR-UC”.

As used herein, the term “biological sample” means any biological material from which nucleic acid and/or protein molecules can be prepared. In various embodiments, the sample from the subject can be obtained either through surgical biopsy or surgical resection. Alternatively, a sample can be obtained through primary patient derived cell lines, or archived patient samples in the form of FFPE (Formalin fixed, paraffin embedded) samples, or fresh frozen samples. Non-limiting examples of “biological sample” include whole blood, peripheral blood, plasma, mucus, urine, semen, lymph, fecal extract, sputum serum, saliva, cheek swab, cells or other bodily fluid or tissue.

“SNP” as used herein is an abbreviation of single nucleotide polymorphism.

“Risk variant” as used herein refers to an allele, whose presence is associated with an increase in susceptibility to an inflammatory bowel disease, including but not limited to Crohn's Disease, Ulcerative Colitis and Medically Refractory-Ulcerative Colitis, relative to an individual who does not have the risk variant.

As used herein, the term “IFNG” refers to the gene encoding IFN-gamma. Similarly, “IFNG production,” or “IFNG secretion” refers to the product expressed from the IFNG genetic locus.

“Treatment” as used herein refer to both therapeutic treatment and prophylactic or preventative measures, wherein the object is to prevent or slow down (lessen) the targeted pathologic condition, prevent the pathologic condition, pursue or obtain good overall survival, or lower the chances of the individual developing the condition even if the treatment is ultimately unsuccessful. Those in need of treatment include those already with the condition as well as those prone to have the condition or those in whom the condition is to be prevented.

“IFNG therapy” or “anti-IFNG therapy” as used herein refers to any reagents that suppress responses to IFNG and/or inhibit IFNG signaling, including, without limitation, inhibition of any molecular signaling step from the IFNG ligand through its receptor to various upstream and/or downstream molecular targets. The anti-IFNG therapy can include the use of a small molecule; a nucleic acid such as siRNA, shRNA, and miRNA; a nucleic acid analogue such as PNA, pc-PNA, and LNA; an aptamer; a ribosome; a peptide; a protein; an avimer; an antibody, or variants and fragments thereof; and/or combinations of any thereof.

An example of SNPs rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, and/or rs1861487 are provided herein as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, and SEQ ID NO: 37, respectively.

Described herein are methods of treating inflammatory bowel disease using anti-IFNG therapy.

As disclosed herein, the inventors have associated IFNG loci with IBD, CD and UC (Jostins et al., 2012. Nature. 491:119-124, which is herein incorporated by reference as though fully set forth). An association was also found with MR-UC patients. Non-MR-UC and MR-UC samples were compared and an association of MR-UC was observed with anti-microbial antibody levels (ASCA-IgA, CBIR1 and OMPC levels). Methylation levels in UC and CD patients were analyzed and demonstrate that IFNG DNA methylation inversely correlates with IFNG secretion in UC. Furthermore, a decreased IFNG methylation level is associated with increased antibody reactivity to microbial antigens in UC.

The IFNG gene has conserved regions between human and mouse and the IFNG+2109 SNP rs1861494 was found to be located in a conserved regulatory region of the third intron of IFNG (Gonsky et al., Inflamm Bowel Dis. 2014. 20:1794-1801, which is herein incorporated by reference as though fully set forth). In particular, individuals carrying the C allele at the methylation site of rs1861494 demonstrate a decrease in IFNG secretion. IBD patients heterozygous for re1861494 demonstrate allele specific methylation.

The inventors have identified IFNG associated SNPs and methylation patterns in IBD, CD, UC and/or MR-UC.

The present invention is based, at least in part, on these findings. The present invention addresses the need in the art for methods of treating IBD, CD, UC and/or MR-UC using anti-IFNG therapy. The invention further provides a process for identifying a subject in need of IFNG therapy.

Fontolizumab (available from Abbvie, Inc.) is an IFNG inhibitor, which can be used as an anti-IFNG therapy for IBD, CD, UC and/or MR-UC in accordance with various embodiments of the present invention.

### Methods of Treatment

Various embodiments of the present invention provide for a method for treating inflammatory disorders of the gastrointestinal tract using anti-IFNG therapy.

In various embodiments, the present invention provides a method of treating, preventing, reducing the severity of, reducing the likelihood of developing and/or reducing the likelihood of recurrence of inflammatory bowel disease in a subject, comprising administering a therapeutically effective amount of said anti-IFNG therapy to the subject. In various embodiments, the method comprises providing an anti-IFNG therapy; and administering a therapeutically effective amount of said anti-IFNG therapy to the subject. In some embodiments, the anti-IFNG therapy comprises fontolizumab. In other embodiments, the inflammatory bowel disease is ulcerative colitis. In yet other embodiments, the inflammatory bowel disease is MR-UC. In various embodiments, the subject has been diagnosed with MR-UC. In various other embodiments, the subject has been diagnosed with the presence of one or more IFNG risk variants. In various other embodiments, the subject has been diagnosed with the presence of 2, 3, 4, 5, 6, 7, 8, 9, 10, 11, 12, 13, 14, 15, 16, 17, 18, 19, 20, 21, 22, 23, 24, 25, 26, 27, 28, 29, 30, 21, 32, 33, 34, 35, 36 or 37 IFNG risk variants as described herein. In certain embodiments, the one or more risk variants are rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 or a combination thereof (Table 1). In other embodiments, the one or more risk variants are rs1861494, rs7134599, rs1558744 or a combination thereof. In yet other embodiments, the risk variant is rs1861494.

TABLE 1
IFNG Risk Variants

| rs ID # | SEQ ID NO: |
|---|---|
| rs1861494 | 1 |
| rs12318183 | 2 |
| rs1558743 | 3 |
| rs7134599 | 4 |
| rs11614309 | 5 |
| rs12822844 | 6 |
| rs35246047 | 7 |
| rs7138407 | 8 |
| rs7134472 | 9 |
| rs723403 | 10 |
| rs12831020 | 11 |
| rs34902013 | 12 |
| rs12811446 | 13 |
| rs12825700 | 14 |
| rs12815372 | 15 |
| rs11610754 | 16 |
| rs4255613 | 17 |
| rs10878749 | 18 |
| rs1558744 | 19 |
| rs2870955 | 20 |
| rs201251289 | 21 |
| rs7137158 | 22 |
| rs7301797 | 23 |
| rs7306440 | 24 |
| rs722749 | 25 |
| rs1005048 | 26 |
| rs722748 | 27 |
| rs11177053 | 28 |
| rs2111057 | 29 |
| rs11177059 | 30 |
| rs11177050 | 31 |
| rs11177049 | 32 |
| rs7304878 | 33 |
| rs11610401 | 34 |
| rs11614178 | 35 |
| rs11177060 | 36 |
| rs1861487 | 37 |

Various embodiments of the present invention provide for a method for treating inflammatory disorders of the gastrointestinal tract by administering anti-IFNG therapy in a subject. In various embodiments, the inflammatory disorders of the gastrointestinal tract are IBD, CD, UC, and in particular MR-UC. In various embodiments, the subject has been diagnosed with IBD, CD, UC, and/or MR-UC. In some embodiments, the IFNG therapy comprises an immunosuppressive drug. In other embodiments, the IFNG therapy comprises an anti-IFNG antibody. In other embodiments, the IFNG therapy comprises a humanized anti-IFNG antibody. In yet other embodiments, the IFNG therapy comprises fontolizumab.

Subject Identification and/or Stratification

Various embodiments of the present invention provide for a process of identifying a subject in need of IFNG therapy, comprising obtaining a biological sample from a subject; analyzing the sample for the presence or absence of one or more genetic risk variants; and identifying the subject in need of IFNG therapy if one or more genetic risk variants are present. In various embodiments, the method comprises identifying the subject in need of IFNG therapy if 2, 3, 4, 5, 6, 7, 8, 9, 10, 11, 12, 13, 14, 15, 16, 17, 18, 19, 20, 21, 22, 23, 24, 25, 26, 27, 28, 29, 30, 21, 32, 33, 34, 35, 36 or 37 IFNG risk variants as described herein are present. In other embodiments, the subject has been diagnosed with IBD, CD, UC and/or MR-UC. In certain embodiments, the subject has been diagnosed with MR-UC. In certain embodiments, the one or more genetic risk variants are rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 or a combination thereof. In some embodiments, the one or more genetic risk variants are rs1861494, rs7134599, rs1558744 or a combination thereof. In yet other embodiments, the risk variant is rs1861494.

In some other embodiments, the process further comprises treating the subject identified with IFNG therapy. In some embodiments, the IFNG therapy comprises an immunosuppressive drug. In other embodiments, the IFNG therapy comprises an anti-IFNG antibody. In other embodiments, the IFNG therapy comprises a humanized anti-IFNG antibody. In yet other embodiments, the IFNG therapy comprises fontolizumab.

Various embodiments of the present invention can also provide for a process of patient risk stratification to identify the subject in need of IFNG therapy. In various embodiments, the patient is stratified based on the detection of IFNG risk variants in a biological sample from the subject. In some embodiments, the detection of the risk variants in the biological sample stratifies the subject into a group needing IFNG therapy. In various other embodiments, the presence of a greater number of risk variants in the sample can indicate that the subject is in greater need for anti-IFNG therapy. In various embodiments, the detection of the IFNG risk variants can provide a guide for treatment in a subject, wherein the presence of the risk variants is indicative of the need for treatment. In some embodiments, the subject is treated by administering anti-IFNG therapy. In other embodiments, the anti-IFNG therapy comprises fontolizumab.

“Patient Stratification” as used herein means the process of separating subjects into risk groups in need of IFNG therapy.

In various embodiments, the detection of IFNG risk variants can be accomplished by analyzing nucleic acids of a biological sample from the subject. A variety of apparatuses and/or methods can be used, including, without limitation, polymerase chain reaction based analysis, sequence analysis and electrophoretic analysis can be used to detect IFNG risk variants. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

In other embodiments, the treatment method and/or the process for subject identification and/or stratification described herein can further comprise assaying the sample to detect an increase or decrease of at least one risk serological marker and/or the IFNG methylation level, relative to a healthy individual. In various other embodiments, a subject with a decreased level of IFNG methylation is treated with anti-IFNG therapy. In yet other embodiments, the subject with a decreased level of IFNG methylation has an increased level of IFNG secretion. In certain other embodiments, a decrease in IFNG methylation is associated with an increase in antibody reactivity to microbial antigens (serological factors). In some embodiments, the microbial antigens (serological factors) are ANCA, ASCA, OmpC, I2, CBir or a combination thereof. In other embodiments, a subject with an increase in serological factors is treated with anti-IFNG therapy. In yet other embodiments, a subject with a decrease in IFNG methylation and an increase in serological factors is treated with anti-IFNG therapy. In some embodiments, a subject with the presence of one or more IFNG risk variants, a decrease in IFNG methylation, an increase in serological factors, or a combination thereof, is treated with anti-IFNG therapy. In a further embodiment, the subject can be treated by conducting colectomy and/or administering anti-IFNG therapy. In other embodiments, the anti-IFNG therapy comprises fontolizumab.

### Treatment Administration and Dosage

In various other embodiments, the anti-IFNG therapy is administered in a therapeutically effective amount. In various embodiments, the anti-IFNG therapy is administered to a subject diagnosed with IBD, CD, UC and/or MR-UC. In some embodiments, the anti-IFNG therapy is administered to a subject diagnosed with MR-UC. In various embodiments, the anti-IFNG therapy is used for the treatment of the subject diagnosed with IBD, CD, UC and/or MR-UC. In yet other embodiments, anti-IFNG therapy is used for treatment of the subject diagnosed with MR-UC.

In various embodiments, the anti-IFNG therapy according to the invention may be delivered via various routes of administration. “Route of administration” may refer to any administration pathway known in the art, including but not limited to aerosol, nasal, oral, transmucosal, transdermal or parenteral.

“Transdermal” administration may be accomplished using a topical cream or ointment or by means of a transdermal patch.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the anti-IFNG therapy may be in the form of solutions or suspensions for infusion or for injection, or as lyophilized powders.

Via the enteral route, the anti-IFNG therapy can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the anti-IFNG therapy may be in the form of solutions or suspensions for infusion or for injection.

The anti-IFNG therapy according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

In some embodiments, the IFNG therapy comprises an immunosuppressive drug. In other embodiments, the IFNG therapy comprises an anti-IFNG antibody. In other embodiments, the IFNG therapy comprises a humanized anti-IFNG antibody. In yet other embodiments, the IFNG therapy comprises fontolizumab.

Typical dosages of an effective amount of IFNG therapy can be in the ranges recommended by the manufacturer where known therapeutic compounds are used, and also as indicated to the skilled artisan by the in vitro responses in cells or in vivo responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied diseased tissue, or responses observed in the appropriate animal models.

In various embodiments, the IFNG therapy is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In various embodiments, the IFNG therapy may be administered once a day, twice a day, three times a day, four times a day, or more, or once a week, twice a week, once every two weeks, once every three weeks or once a month, so as to administer an effective amount of the IFNG therapy to the individual, where the effective amount is any one or more of the doses described herein. In various other embodiments, the IFNG therapy is administered once, twice, three or more times. In some embodiments, the IFNG therapy is administered about 1-7 times per week or 1-15 times per month. Still in some embodiments, the IFNG therapy is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. Here, “mg/kg” refers to mg per kg body weight of the individual. In certain embodiments, the IFNG therapy is administered to a human.

In accordance with the invention, the IFNG therapy may be administered using the appropriate modes of administration, for instance, the modes of administration recommended by the manufacturer. In accordance with the invention, various routes may be utilized to administer the IFNG therapy of the claimed methods, including but not limited to aerosol, nasal, oral, transmucosal, transdermal, parenteral, enteral, topical, local, implantable pump, continuous infusion, capsules and/or injections. In various embodiments, the IFNG therapy is administered topically, intravascularly, intravenously, intraarterially, intramuscularly, subcutaneously, intraperitoneally, intranasally, or orally.

### Biological Samples

In various embodiments, the biological sample comprises a nucleic acid from the individual. In some embodiments, the sample comprises a body fluid, cheek swab, mucus, whole blood, blood, serum, plasma, urine, saliva, semen, lymph, fecal extract, or sputum, or a combination thereof. In other embodiments, the sample comprises a cell or tissue.

In various embodiments, the steps involved in the current invention comprise obtaining either through surgical biopsy or surgical resection, a sample from the subject. Alternatively, a sample can be obtained through primary patient derived cell lines, or archived patient samples in the form of FFPE (Formalin fixed, paraffin embedded) samples, or fresh frozen samples.

In various embodiments, the subject is a human. In some embodiments, the subject is a mammalian subject including but not limited to human, monkey, ape, dog, cat, cow, horse, goat, pig, rabbit, mouse and rat.

The subjects sample is then used to extract nucleic acid (Ribonucleic acid (RNA), Deoxyribonucleic acid (DNA)) or protein, using standard protocols well-known in the art.

### Sample Preparation and Gene Expression Detection

Nucleic acid or protein samples derived from diseased and non-diseased cells of a subject that can be used in the methods of the invention can be prepared by means well known in the art. For example, surgical procedures or needle biopsy aspiration can be used to collect diseased samples from a subject. In some embodiments, it is important to enrich and/or purify the diseased tissue and/or cell samples from the non-diseased tissue and/or cell samples. In other embodiments, the diseased tissue and/or cell samples can then be microdissected to reduce the amount of non-diseased tissue contamination prior to extraction of genomic nucleic acid or pre-RNA for use in the methods of the invention. Such enrichment and/or purification can be accomplished according to methods well-known in the art, such as needle microdissection, laser microdissection, fluorescence activated cell sorting, and immunological cell sorting.

Analysis of the nucleic acid and/or protein from an individual may be performed using any of various techniques. In various embodiments, assaying gene expression levels for IFNG comprises northern blot, reverse transcription PCR, real-time PCR, serial analysis of gene expression (SAGE), DNA microarray, SNP array, tiling array, RNA-Seq, or a combination thereof.

In various embodiments, methods and systems to detect protein expression include but are not limited to ELISA, immunohistochemistry, western blot, flow cytometry, fluorescence in situ hybridization (FISH), radioimmuno assays, and affinity purification.

In various embodiments, IFNG and/or the microbial antigen responses are assessed by ELISA. In various other embodiments, protein-DNA and/or protein-RNA interactions are analyzed using electrophoretic mobility shift assays (EMSA).

As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The analysis of gene expression levels may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

A DNA sample suitable for hybridization can be obtained, e.g., by polymerase chain reaction (PCR) amplification of genomic DNA, fragments of genomic DNA, fragments of genomic DNA ligated to adaptor sequences or cloned sequences. Computer programs that are well known in the art can be used in the design of primers with the desired specificity and optimal amplification properties, such as Oligo version 5.0 (National Biosciences). PCR methods are well known in the art, and are described, for example, in Innis et al., eds., 1990, PCR Protocols: A Guide to Methods And Applications, Academic Press Inc., San Diego, Calif. It will be apparent to one skilled in the art that controlled robotic systems are useful for isolating and amplifying nucleic acids and can be used.

### Hybridization

The nucleic acid samples derived from a subject used in the methods of the invention can be hybridized to arrays comprising probes (e.g., oligonucleotide probes) in order to identify IFNG and in instances wherein a housekeeping gene expression is also to be assessed, comprising probes in order to identify housekeeping genes. In particular embodiments, the probes used in the methods of the invention comprise an array of probes that can be tiled on a DNA chip (e.g., SNP oligonucleotide probes). Hybridization and wash conditions used in the methods of the invention are chosen so that the nucleic acid samples to be analyzed by the invention specifically bind or specifically hybridize to the complementary oligonucleotide sequences of the array, preferably to a specific array site, wherein its complementary DNA is located. In some embodiments, the complementary DNA can be completely matched or mismatched to some degree as used, for example, in Affymetrix oligonucleotide arrays. The single-stranded synthetic oligodeoxyribonucleic acid DNA probes of an array may need to be denatured prior to contact with the nucleic acid samples from a subject, e.g., to remove hairpins or dimers which form due to self-complementary sequences.

Optimal hybridization conditions will depend on the length of the probes and type of nucleic acid samples from a subject. General parameters for specific (i.e., stringent) hybridization conditions for nucleic acids are described in Sambrook and Russel, Molecular Cloning: A Laboratory Manual 4^{th} ed., Cold Spring Harbor Laboratory Press (Cold Spring Harbor, N.Y. 2012); Ausubel et al., eds., 1989, Current Protocols in Molecules Biology, Vol. 1, Green Publishing Associates, Inc., John Wiley & Sons, Inc., New York, at pp. 2.10.1-2.10.16. Exemplary useful hybridization conditions are provided in, e.g., Tijessen, 1993, Hybridization with Nucleic Acid Probes, Elsevier Science Publishers B. V. and Kricka, 1992, Nonisotopic DNA Probe Techniques, Academic Press, San Diego, Calif.

### Oligonucleotide Nucleic Acid Arrays

In some embodiments of the methods of the present invention, DNA arrays can be used to determine the expression levels of genes, by measuring the level of hybridization of the nucleic acid sequence to oligonucleotide probes that comprise complementary sequences. Various formats of DNA arrays that employ oligonucleotide “probes,” (i.e., nucleic acid molecules having defined sequences) are well known to those of skill in the art. Typically, a set of nucleic acid probes, each of which has a defined sequence, is immobilized on a solid support in such a manner that each different probe is immobilized to a predetermined region. In certain embodiments, the set of probes forms an array of positionally-addressable binding (e.g., hybridization) sites on a support. Each of such binding sites comprises a plurality of oligonucleotide molecules of a probe bound to the predetermined region on the support. More specifically, each probe of the array is preferably located at a known, predetermined position on the solid support such that the identity (i.e., the sequence) of each probe can be determined from its position on the array (i.e., on the support or surface). Microarrays can be made in a number of ways, of which several are described herein. However produced, microarrays share certain characteristics, they are reproducible, allowing multiple copies of a given array to be produced and easily compared with each other.

In some embodiments, the microarrays are made from materials that are stable under binding (e.g., nucleic acid hybridization) conditions. The microarrays are preferably small, e.g., between about 1 cm² and 25 cm², preferably about 1 to 3 cm². However, both larger and smaller arrays are also contemplated and may be preferable, e.g., for simultaneously evaluating a very large number of different probes. Oligonucleotide probes can be synthesized directly on a support to form the array. The probes can be attached to a solid support or surface, which may be made, e.g., from glass, plastic (e.g., polypropylene, nylon), polyacrylamide, nitrocellulose, gel, or other porous or nonporous material. The set of immobilized probes or the array of immobilized probes is contacted with a sample containing labeled nucleic acid species so that nucleic acids having sequences complementary to an immobilized probe hybridize or bind to the probe. After separation of, e.g., by washing off, any unbound material, the bound, labeled sequences are detected and measured. The measurement is typically conducted with computer assistance. DNA array technologies have made it possible to determine the expression level of IFNG and housekeeping genes, as mentioned above.

In certain embodiments, high-density oligonucleotide arrays are used in the methods of the invention. These arrays containing thousands of oligonucleotides complementary to defined sequences, at defined locations on a surface can be synthesized in situ on the surface by, for example, photolithographic techniques (see, e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; 5,510,270; 5,445,934; 5,744,305; and 6,040,138). Methods for generating arrays using inkjet technology for in situ oligonucleotide synthesis are also known in the art (see, e.g., Blanchard, International Patent Publication WO 98/41531, published Sep. 24, 1998; Blanchard et al., 1996, Biosensors And Bioelectronics 11:687-690; Blanchard, 1998, in Synthetic DNA Arrays in Genetic Engineering, Vol. 20, J. K. Setlow, Ed., Plenum Press, New York at pages 111-123). Another method for attaching the nucleic acids to a surface is by printing on glass plates, as is described generally by Schena et al. (1995, Science 270:467-470). Other methods for making microarrays, e.g., by masking (Maskos and Southern, 1992, Nucl. Acids. Res. 20:1679-1684), may also be used. When these methods are used, oligonucleotides (e.g., 15 to 60-mers) of known sequence are synthesized directly on a surface such as a derivatized glass slide. The array produced can be redundant, with several oligonucleotide molecules corresponding to each informative locus of interest (e.g., SNPs, RFLPs, STRs, etc.).

One exemplary means for generating the oligonucleotide probes of the DNA array is by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 15 and about 600 bases in length, more typically between about 20 and about 100 bases, most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., 1993, Nature 363:566-568; U.S. Pat. No. 5,539,083). In alternative embodiments, the hybridization sites (i.e., the probes) are made from plasmid or phage clones of regions of genomic DNA corresponding to SNPs or the complement thereof. The size of the oligonucleotide probes used in the methods of the invention can be at least 10, 20, 25, 30, 35, 40, 45, or 50 nucleotides in length. It is well known in the art that although hybridization is selective for complementary sequences, other sequences which are not perfectly complementary may also hybridize to a given probe at some level. Thus, multiple oligonucleotide probes with slight variations can be used, to optimize hybridization of samples. To further optimize hybridization, hybridization stringency condition, e.g., the hybridization temperature and the salt concentrations, may be altered by methods that are well known in the art.

In various embodiments, the high-density oligonucleotide arrays used in the methods of the invention comprise oligonucleotides corresponding to IFNG and appropriate housekeeping genes. The oligonucleotide probes may comprise DNA or DNA “mimics” (e.g., derivatives and analogues) corresponding to a portion of each informative locus of interest (e.g., SNPs, RFLPs, STRs, etc.) in a subject's genome. The oligonucleotide probes can be modified at the base moiety, at the sugar moiety, or at the phosphate backbone. Exemplary DNA mimics include, e.g., phosphorothioates. For each SNP locus, a plurality of different oligonucleotides may be used that are complementary to the sequences of sample nucleic acids. For example, for a single informative locus of interest (e.g., SNPs, RFLPs, STRs, etc.) about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more different oligonucleotides can be used. Each of the oligonucleotides for a particular informative locus of interest may have a slight variation in perfect matches, mismatches, and flanking sequence around the SNP. In certain embodiments, the probes are generated such that the probes for a particular informative locus of interest comprise overlapping and/or successive overlapping sequences which span or are tiled across a genomic region containing the target site, where all the probes contain the target site. By way of example, overlapping probe sequences can be tiled at steps of a predetermined base interval, e. g. at steps of 1, 2, 3, 4, 5, 6, 7, 8, 9, or 10 bases intervals. In certain embodiments, the assays can be performed using arrays suitable for use with molecular inversion probe protocols such as described by Wang et al. (2007) Genome Biol. 8, 8246. For oligonucleotide probes targeted at nucleic acid species of closely resembled (i.e., homologous) sequences, “cross-hybridization” among similar probes can significantly contaminate and confuse the results of hybridization measurements. Cross-hybridization is a particularly significant concern in the detection of SNPs since the sequence to be detected (i.e., the particular SNP) must be distinguished from other sequences that differ by only a single nucleotide. Cross-hybridization can be minimized by regulating either the hybridization stringency condition and/or during post-hybridization washings. Highly stringent conditions allow detection of allelic variants of a nucleotide sequence, e.g., about 1 mismatch per 10-30 nucleotides. There is no single hybridization or washing condition which is optimal for all different nucleic acid sequences, these conditions can be identical to those suggested by the manufacturer or can be adjusted by one of skill in the art. In some embodiments, the probes used in the methods of the invention are immobilized (i.e., tiled) on a glass slide called a chip. For example, a DNA microarray can comprises a chip on which oligonucleotides (purified single-stranded DNA sequences in solution) have been robotically printed in an (approximately) rectangular array with each spot on the array corresponds to a single DNA sample which encodes an oligonucleotide. In summary the process comprises, flooding the DNA microarray chip with a labeled sample under conditions suitable for hybridization to occur between the slide sequences and the labeled sample, then the array is washed and dried, and the array is scanned with a laser microscope to detect hybridization. In certain embodiments there are at least 250, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 50,000, 60,000, 70,000, 80,000, 90,000, 100,000 or more or any range in between, of IFNG or the housekeeping genes for which probes appear on the array (with match/mismatch probes for a single locus of interest or probes tiled across a single locus of interest counting as one locus of interest). The maximum number of IFNG or housekeeping genes being probed per array is determined by the size of the genome and genetic diversity of the subject's species. DNA chips are well known in the art and can be purchased in pre-5 fabricated form with sequences specific to particular species. In other embodiments, SNPs and/or DNA copy number can be detected and quantitated using sequencing methods, such as “next-generation sequencing methods”.

### Labeling

In some embodiments, the protein, polypeptide, nucleic acid, fragments thereof, or fragments thereof ligated to adaptor regions used in the methods of the invention are detectably labeled. For example, the detectable label can be a fluorescent label, e.g., by incorporation of nucleotide analogues. Other labels suitable for use in the present invention include, but are not limited to, biotin, iminobiotin, antigens, cofactors, dinitrophenol, lipoic acid, olefinic compounds, detectable polypeptides, electron rich molecules, enzymes capable of generating a detectable signal by action upon a substrate, and radioactive isotopes.

Radioactive isotopes include that can be used in conjunction with the methods of the invention, but are not limited to, 32P and 14C. Fluorescent molecules suitable for the present invention include, but are not limited to, fluorescein and its derivatives, rhodamine and its derivatives, texas red, 5′carboxy-fluorescein (“FAM”), 2′,7′-dimethoxy-4′,5′-dichloro-6-carboxy-fluorescein (“JOE”), N, N, N′,N′-tetramethyl-6-carboxy-rhodamine (“TAMRA”), 6-carboxy-X-rhodamine (“ROX”), HEX, TET, IRD40, and IRD41.

Fluorescent molecules which are suitable for use according to the invention further include: cyamine dyes, including but not limited to Cy2, Cy3, Cy3.5, CY5, Cy5.5, Cy7 and FLUORX; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin and colloidal gold.

Two-color fluorescence labeling and detection schemes may also be used (Shena et al., 1995, Science 270:467-470). Use of two or more labels can be useful in detecting variations due to minor differences in experimental conditions (e.g., hybridization conditions). In some embodiments of the invention, at least 5, 10, 20, or 100 dyes of different colors can be used for labeling. Such labeling would also permit analysis of multiple samples simultaneously which is encompassed by the invention.

The labeled nucleic acid samples, fragments thereof, or fragments thereof ligated to adaptor regions that can be used in the methods of the invention are contacted to a plurality of oligonucleotide probes under conditions that allow sample nucleic acids having sequences complementary to the probes to hybridize thereto. Depending on the type of label used, the hybridization signals can be detected using methods well known to those of skill in the art including, but not limited to, X-Ray film, phosphor imager, or CCD camera. When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al. (1996) Genome Res. 6, 639-645). In a preferred embodiment, the arrays are scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al. (1996) Genome Res. 6, 639-645. Alternatively, a fiber-optic bundle can be used such as that described by Ferguson et al. (1996) Nat. Biotech. 14, 1681-1684. The resulting signals can then be analyzed to determine the expression of IFNG and the reference genes, using computer software.

In other embodiments, where genomic DNA of a subject is fragmented using restriction endonucleases and amplified prior to analysis, the amplification can comprise cloning regions of genomic DNA of the subject. In such methods, amplification of the DNA regions is achieved through the cloning process. For example, expression vectors can be engineered to express large quantities of particular fragments of genomic DNA of the subject (Sambrook and Russel, Molecular Cloning: A Laboratory Manual 4^{th} ed., Cold Spring Harbor Laboratory Press (Cold Spring Harbor, N.Y. 2012)).

In yet other embodiments, where the DNA of a subject is fragmented using restriction endonucleases and amplified prior to analysis, the amplification comprises expressing a nucleic acid encoding a gene, or a gene and flanking genomic regions of nucleic acids, from the subject. RNA (pre-messenger RNA) that comprises the entire transcript including introns is then isolated and used in the methods of the invention to analyze and provide a genetic signature. In certain embodiments, no amplification is required. In such embodiments, the genomic DNA, or pre-RNA, of a subject may be fragmented using restriction endonucleases or other methods. The resulting fragments may be hybridized to SNP probes. Typically, greater quantities of DNA are needed to be isolated in comparison to the quantity of DNA or pre-mRNA needed where fragments are amplified. For example, where the nucleic acid of a subject is not amplified, a DNA sample of a subject for use in hybridization may be about 400 ng, 500 ng, 600 ng, 700 ng, 800 ng, 900 ng, or 1000 ng of DNA or greater. Alternatively, in other embodiments, methods are used that require very small amounts of nucleic acids for analysis, such as less than 400 ng, 300 ng, 200 ng, 100 ng, 90 ng, 85 ng, 80 ng, 75 ng, 70 ng, 65 ng, 60 ng, 55 ng, 50 ng, or less, such as is used for molecular inversion probe (MIP) assays. These techniques are particularly useful for analyzing clinical samples, such as paraffin embedded formalin-fixed material or small core needle biopsies, characterized as being readily available but generally having reduced DNA quality (e.g., small, fragmented DNA) and/or not providing large amounts of nucleic acids.

Once the expression levels have been determined, the resulting data can be analyzed using various algorithms, based on well-known methods used by those skilled in the art.

### Detection of Methylation

Various embodiments provide for a method of treating a subject in need of treatment with anti-IFNG therapy. In some embodiments, the method comprises administering an anti-IFNG therapy to a subject who has a decrease in IFNG methylation. In other embodiments, the method comprises administering an anti-IFNG therapy to a subject who has a decrease in IFNG methylation and who has a presence of one or more risk variants.

Various embodiments provide for a process of identifying a subject in need of treatment with anti-IFNG therapy. In some embodiments, the process comprises assessing the level of IFNG methylation; and identifying the subject who has a decreased level of IFNG methylation as a subject in need to treatment with anti-IFNG therapy. In some embodiments, the process comprises assessing the level of IFNG methylation; assessing the presence or absence of one or more risk variants as disclosed therein; and identifying the subject who has a decreased level of IFNG methylation and one or more risk variants as a subject in need to treatment with anti-IFNG therapy.

Various methods to detect levels of methylation include, but are not limited to the following assays, mass spectrometry, methylation-specific PCR (MSP), whole genome bisulfite sequencing, (BS-Seq), the HELP assay, ChIP-on-chip assays, restriction landmark genomic scanning, methylated DNA immunoprecipitation (MeDIP, MeDIP-chip, MeDIP-seq), pyrosequencing of bisulfite treated DNA, molecular break light assay for DNA adenine methyltransferase activity, methyl sensitive southern blotting, separate native DNA into methylated and unmethylated fractions using MethylCpG Binding Proteins (MBPs) and/or Methyl Binding Domain (MBD), MethylationEPIC BeadChip, Illumina Infinium Methylation 450 BeadChip, High Resolution Melt Analysis (HRM or HRMA), and/or ancient DNA methylation reconstruction.

### Kits

The present invention is also directed to a kit to treat IBD, CD, UC and/or MR-UC in a subject. The kit is useful for practicing the inventive method of providing treatment to an IBD, CD, UC and/or MR-UC patient by administering anti-IFNG therapy. The kit is an assemblage of materials or components, including an anti-IFNG drug, for treatment of IBD, CD, UC and/or MR-UC, as described above.

The exact nature of the components configured in the inventive kit depends on its intended purpose. For example, some embodiments are configured for the purpose of treatment of IBD, CD, UC and/or MR-UC. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals. In other embodiments, the kit is configured to determine the level of IFNG expression and/or methylation.

Instructions for use may be included in the kit. “Instructions for use” typically include a tangible expression describing the technique to be employed in using the components of the kit to effect a desired outcome, such as to treat subject with IBD, CD, UC and/or MR-UC, or to determine the level of IFNG expression and/or methylation. Optionally, the kit also contains other useful components, such as, primers/probes, diluents, buffers, pipetting or measuring tools or other useful paraphernalia as will be readily recognized by those of skill in the art and aids in the use of the kit and its components.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing primers and probes for determining the level of IFNG expression and/or methylation. In another example a package can be a glass vial used to contain suitable quantities of an inventive composition a treat subject with IBD, CD, UC and/or MR-UC. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### IFNG

The IFNG locus has been found to be associated with IBD and UC in international genome wide association studies (GWAS), with UC associated the most significantly (Jostins et al., 2012. Nature. 491:119-124, which is herein incorporated by reference as though fully set forth). An association was also found with MR-UC patients (Table 2). The data support an association of the IFNG locus with MR-UC in Cedars-Sinai patient cohort (Table 2 and 3). MR-UC is further associated with increased quartile sums for anti-microbial antibodies (ASCA-IgA, CBIR1 and OMPC levels). Methylation levels were analyzed and demonstrate that IFNG DNA methylation inversely correlates with IFNG secretion and methylation analysis of the IFNG locus has showed an association between IFNG methylation and increased quartile sums for antibodies in UC patients. The level of IFNG methylation at the IFNG promoter region is correlated with the allele-specific methylation of SNP rs1861494. This SNP is in linkage disequilibrium with a region correlated with the development of severe MR-UC. The IFNG gene shows conserved regions between human and mouse. The IFNG+2109 SNP rs1861494 is located within a conserved regulatory region of the third intron of IFNG (Gonsky et al., Inflamm Bowel Dis. 2014. 20:1794-1801, which is herein incorporated by reference as though fully set forth). These data are indicative of the usefulness of an anti-IFNG therapy for treatment of a population of MR-UC patients.

TABLE 3
IFNG LOCI Association with IBD, Crohn's Disease and Ulcerative Colitis

| Chr | Pos_hg19 (Mb) | GWAS_SNP | GWAS_risk | GWAS_nonrisk | MostSig | p_ALL_CD | P_ALL_UC | p_ALL_IBD |
|---|---|---|---|---|---|---|---|---|
| 12 | 68.21-68.74 | rs7134599 | A | G | UC | 4.16E−05 | 8.51E−32 | 1.22E−22 |
| | GWAS_SNP | MAF_IC | OR_CD | 95% Cl | OR_UC | 95% Cl | OR_IBD | 95% Cl |
| | rs7134599 | 0.389 | 1.053 | 1.018-1.088 | 1.156 | 1.115-1.197 | 1.096 | 1.064-1.128 |

### Example 2

A patient diagnosed as having MR-UC is provided with two doses of an IFNG therapy and instructed to administer a single dose of IFNG therapy every 28 days. The patient is assessed at two week intervals up to day 56, and monthly thereafter for a year. The assessments include hematology and chemistry panels, immunogenicity, CDAI scores, fontolizumab pharmacokinetics and any adverse events will be noted. The presence or absence of fontolizumab and/or IFNG are assessed via ELISA. Samples assessed are collected before and after treatment and every two weeks thereafter.

### Example 3

A patient diagnosed as having MR-UC is given intravenous fontolizumab (4 or 10 mg/kg) infused over 30 minutes for two doses, 28 days apart.

The patient is assessed at two week intervals up to day 56, and monthly thereafter for a year. The assessments include hematology and chemistry panels, immunogenicity, CDAI scores, fontolizumab pharmacokinetics and any adverse events will be noted. The presence or absence of fontolizumab and/or IFNG are assessed via ELISA. Samples assessed are collected before and after treatment and every two weeks thereafter.

### Example 4

A patient diagnosed with the presence of one or more risk variants, where the one or more risk variants are rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 or a combination thereof, is provided with two doses of an IFNG therapy and instructed to administer a single dose of IFNG therapy every 28 days.

The patient is assessed at two week intervals up to day 56, and monthly thereafter for a year. The assessments include hematology and chemistry panels, immunogenicity, CDAI scores, fontolizumab pharmacokinetics and any adverse events will be noted. The presence or absence of fontolizumab and/or IFNG is assessed via ELISA. Samples assessed are collected before and after treatment and every two weeks thereafter.

### Example 5

A patient diagnosed as having one or more risk variants, where the one or more risk variants are rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 and/or a combination thereof, is given intravenous fontolizumab (4 or 10 mg/kg) infused over 30 minutes for two doses, 28 days apart.

The patient is assessed at two week intervals up to day 56, and monthly thereafter for a year. The assessments include hematology and chemistry panels, immunogenicity, CDAI scores, fontolizumab pharmacokinetics and any adverse events will be noted. The presence or absence of fontolizumab and/or IFNG is assessed via ELISA. Samples assessed are collected before and after treatment and every two weeks thereafter.

### Example 6

A patient diagnosed as having MR-UC is provided with multiple doses of an IFNG therapy and instructed to administer a single dose of IFNG therapy every 28 days, until amelioration of the disease and/or disease symptoms.

The patient is assessed at two week intervals up to day 56, and monthly thereafter for a year or until amelioration of the disease and/or disease symptoms. The assessments include hematology and chemistry panels, immunogenicity, CDAI scores, fontolizumab pharmacokinetics and any adverse events will be noted. The presence or absence of fontolizumab and/or IFNG are assessed via ELISA. Samples assessed are collected before and after treatment and every two weeks thereafter.

### Example 7

A patient diagnosed as having MR-UC is given intravenous fontolizumab (4 or 10 mg/kg) infused over 30 minutes for multiple doses, 28 days apart, until amelioration of the disease and/or disease symptoms.

The patient is assessed at two week intervals up to day 56, and monthly thereafter for a year or until amelioration of the disease and/or disease symptoms. The assessments include hematology and chemistry panels, immunogenicity, CDAI scores, fontolizumab pharmacokinetics and any adverse events will be noted. The presence or absence of fontolizumab and/or IFNG are assessed via ELISA. Samples assessed are collected before and after treatment and every two weeks thereafter.

### Example 8

A patient diagnosed with the presence of one or more risk variants, where the one or more risk variants are rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 or a combination thereof, is provided with multiple doses of an IFNG therapy and instructed to administer a single dose of IFNG therapy every 28 days, until amelioration of the disease and/or disease symptoms.

The patient is assessed at two week intervals up to day 56, and monthly thereafter for a year or until amelioration of the disease and/or disease symptoms. The assessments include hematology and chemistry panels, immunogenicity, CDAI scores, fontolizumab pharmacokinetics and any adverse events will be noted. The presence or absence of fontolizumab and/or IFNG are assessed via ELISA. Samples assessed are collected before and after treatment and every two weeks thereafter.

### Example 9

A patient diagnosed with the presence of one or more risk variants, where the one or more risk variants are rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 or a combination thereof, is given intravenous fontolizumab (4 or 10 mg/kg) infused over 30 minutes for multiple doses, 28 days apart, until amelioration of the disease and/or disease symptoms.

The patient is assessed at two week intervals up to day 56, and monthly thereafter for a year or until amelioration of the disease and/or disease symptoms. The assessments include hematology and chemistry panels, immunogenicity, CDAI scores, fontolizumab pharmacokinetics and any adverse events will be noted. The presence or absence of fontolizumab and/or IFNG are assessed via ELISA. Samples assessed are collected before and after treatment and every two weeks thereafter.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method of treating, preventing, reducing the severity of, reducing the likelihood of developing and/or reducing the likelihood of recurrence of inflammatory bowel disease in a subject, comprising:
administering a therapeutically effective amount of said anti-IFNG therapy to the subject.

2. The method of claim 1, wherein the anti-IFNG therapy comprises fontolizumab.

3. The method of claim 1, wherein the inflammatory bowel disease is ulcerative colitis.

4. The method of claim 1, wherein the inflammatory bowel disease is medically refractory ulcerative colitis.

5. The method of claim 1, wherein the subject has been diagnosed with MR-UC.

6. The method of claim 1, wherein the subject has been diagnosed with one or more risk variants selected from the group consisting of rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 and a combination thereof.

7. The method of claim 1, wherein the subject has an increase in IFNG secretion.

8. The method of claim 1, wherein the subject has a decrease in IFNG methylation.

9. The method of claim 1, wherein the subject has an increased level of a serological factor.

10. The method of claim 9, wherein the serological factor is ANCA, ASCA, OmpC, I2, CBir or a combination thereof.

11. A process of identifying a subject in need of IFNG therapy, and optionally treating the subject, comprising:
obtaining a biological sample from a subject;
analyzing the sample for the presence or absence of one or more genetic risk variants; and
identifying the subject in need of IFNG therapy if one or more genetic risk variants are present.

12. The process of claim 11, wherein the subject has been diagnosed with IBD, CD and/or UC.

13. The process of claim 11, wherein the subject has been diagnosed with MR-UC.

14. The process of claim 11, wherein the one or more genetic risk variants is selected from the group consisting of rs1861494, rs12318183, rs1558743, rs7134599, rs11614309, rs12822844, rs35246047, rs7138407, rs7134472, rs723403, rs12831020, rs34902013, rs12811446, rs12825700, rs12815372, rs11610754, rs4255613, rs10878749, rs1558744, rs2870955, rs201251289, rs7137158, rs7301797, rs7306440, rs722749, rs1005048, rs722748, rs11177053, rs2111057, rs11177059, rs11177050, rs11177049, rs7304878, rs11610401, rs11614178, rs11177060, rs1861487 and a combination thereof.

15. The process of claim 11, further comprising treating the subject identified with IFNG therapy.

16. The process of claim 15, wherein the IFNG therapy comprises fontolizumab.

17. The process of claim 11, wherein the subject has an increase in IFNG secretion.

18. The process of claim 11, wherein the subject has a decrease in IFNG methylation.

19. The process of claim 11, wherein the subject has an increase in antibody reactivity to a serological factor.

20. The method of claim 19, wherein the serological factor is ANCA, ASCA, OmpC, I2, CBir or a combination thereof.

21. A pharmaceutical composition for treating, preventing, reducing the severity of, reducing the likelihood of developing and/or reducing the likelihood of recurrence of inflammatory bowel disease in a subject, comprising:
an anti-IFNG therapy; and
a pharmaceutically acceptable carrier.
